(19) United States
(12) Patent Application Publication
(10) Pub. No.: US 2004/0102364 A1
(43) Pub. Date: May 27, 2004

(54) Backbone cyclized radiolabelled somatostatin analogs

(76) Inventors: Thomas A. Bonasera, Milano (IT); Nurit Livnah, Mazkeret Batya (IL); Tamar Yechezkel, Ramat-Gan (IL); Yoseph Salitra, Rehovot (IL)

Correspondence Address: WINSTON & STRAWN, PATENT DEPARTMENT, 1400 L STREET, N.W., WASHINGTON, DC, 20005-3502, US

(21) Appl. No.: 10/634,496

(22) Filed: Aug. 4, 2003

Related U.S. Application Data
(63) Continuation of application No. PCT/IL02/00091, filed on Feb. 4, 2002.

(30) Foreign Application Priority Data
Feb. 5, 2001 (IL) 141276

(51) Int. Cl. A61K 38/12; C07K 14/665

(52) U.S. Cl. 514/9; 530/311

(57) ABSTRACT

Novel radiodiagnostic and radiotherapeutic peptides which are conformationally constrained backbone cyclized somatostatin analogs, having improved somatostatin receptor subtype affinity and selectivity are disclosed. The backbone cyclized peptide analogs disclosed possess unique and superior properties over other analogs, such as chemical and metabolic stability, selectivity, increased bioavailability and improved pharmacokinetics. Furthermore, the unique patterns of receptor subtype selectivity provide compounds having improved diagnostic and therapeutic utilities. Pharmaceutical compositions comprising the backbone cyclized somatostatin analogs and radiolabelled analogs, reagents for synthesizing same, and methods of using such compositions for radiodiagnostic and radiotherapeutic purposes are also disclosed.

### CROSS-REFERENCE TO RELATED APPLICATIONS

This application is a continuation of International application PCT/IL02/00091 filed Feb. 4, 2002, the entire content of which is expressly incorporated herein by reference thereto.

### FIELD OF THE INVENTION

The present invention relates to radiolabelled somatostatin analogs which are conformationally constrained N^{α} backbone-cyclized peptides, to pharmaceutical compositions containing same, to reagents for synthesizing same, and to methods for using such compounds for diagnosis and therapy. Within the scope of the present invention certain novel somatostatin analogs are disclosed and claimed as such.

### BACKGROUND OF THE INVENTION

Somatostatin (SST) is a cyclic tetradecapeptide found both in the central nervous system and in peripheral tissues. It was originally isolated from mammalian hypothalamus and identified as an important inhibitor of growth hormone secretion from the anterior pituitary. Its multiple biological activities include inhibition of the secretion of glucagon and insulin from the pancreas, regulation of most gut hormones and regulation of the release of other neurotransmitters involved in motor activity and cognitive processes throughout the central nervous system (for review see Lamberts, Endocrine Rev., 9:427, 1988). Additionally, SST and its analogs are potentially useful antiproliferative agents for the treatment of various types of tumors. Natural SST (also known as Somatotropin Release Inhibiting Factor, SRIF) having the following structure:

H-Ala¹-Gly²-Cys³-Lys⁴-Asn⁵-Phe⁶-Phe⁷-Trp⁸-Lys⁹-Thr¹⁰-Phe¹¹-Thr¹²-Ser¹³-Cys¹⁴-OH was first isolated by Guillemin and colleagues (Bruzeau et al. Science, 179:78, 1973). The two cysteines form a disulfide bridge, and the Phe⁷-Trp⁸-Lys⁹-Thr¹⁰ moiety is considered an essential pharmacophore of the molecule (the residue numbering follows the original SST numbering). In its natural form, SST has limited use as a therapeutic agent since it exhibits two undesirable properties: poor bioavailability and short duration of action. For these reasons, great efforts have been made to find SST analogs that will have superiority in potency, biostability, duration of action or selectivity.

The diverse physiological effects of SST are induced by selective and high affinity binding to receptors that are members of the seven transmembrane segment receptor superfamily (reviewed in Reisine T., Bell G I., Endocrinology Rev., 16:427-442. 1995). So far, five SST receptor subtypes have been isolated and cloned designated SST-R1 through SST-R5. These receptors are characterized by a high degree of sequence homology, but are linked to different multiple cellular effector systems. The receptor subtypes recognize both naturally-occurring and synthetic ligands with different affinities. The receptor subtypes are expressed in a tissue-specific manner and in different tissues they couple to different signal-transduction pathways.

Therapeutic Uses of Somatostatin Analogs

By virtue of their inhibitory pharmacological properties, SST analogs can be used for the treatment of patients with hormone-secreting and hormone-dependent tumors. Octreotide (U.S. Pat. Nos. 4,310,518 and 4,235,886) was the first SST analog approved for clinical use. Symptoms associated with metastatic carcinoid tumors and vasoactive intestinal peptide secreting adenomas are treated with Octreotide. Octreotide was also approved for the treatment of severe gastrointestinal hemorrhages and acromegaly. In neuroendocrine tumors, particularly carcinoids and VIPomas, Octreotide inhibits both the secretion and the effect of the active agent. However, response to Octreotide often decreases with time, possibly due to down-regulation of SST receptors on tumor cells or to the generation of receptor negative clone. The absence of consistent antiproliferative effect may be related to the poor affinity of Octreotide to some of the SST receptor subtypes found in these tumors.

Somatostatin in Cancer

Because SST receptors are present in high density in many endocrine and non-endocrine tumors, diagnosis and treatment were attempted using radiolabelled SST analogs in cancer patients. Most tumors express multiple SST receptor-subtypes, although the SST-R2 subtype is most predominantly expressed. Radiolabelled receptor-specific compounds can detect primary sites, identify occult metastatic lesions, guide surgical intervention, stage tumors, predict efficacy of certain therapeutic agents or, when labelled with suitable radionuclides, be useful radiotherapeutic agents. High-affinity SST receptors have been found to be abundantly expressed at the cell surface of most endocrine-active tumors and serve as markers for these tumor cells. The abundance of high affinity SST receptors in various tumors enables the use of radio-labeled SST analogs for in vivo identification, visualization and localization of these tumors (Lamberts et al. N. Engl. J. Med., 334:246 1996). Binding studies and autoradiography using radiolabelled SST or its analogs, have shown that 80-90% of all neuroendocrine tumors of the gastrointestinal tract possess high numbers of SST receptors (Reubi et al., J. Steroid Biochem Mol Biol 37: 1073, 1990). It was demonstrated that carcinoid tumors possesses multiple SST receptor subtypes and that SST analogs such as Octreotide, which preferentially bind to receptor type 2 and 5, can be used in diagnosis and medical treatment of these tumors (Nilsson et al., Br J Cancer, 77:632, 1998). Based on binding studies of the cloned receptors, SST-R2 has been suggested to be the main target for Octreotide and a prerequisite for tumor imaging.

Radiolabelled Somatostatin Analogs as Diagnostic/Therapeutic Agents

Scintigraphy using radiolabelled SST analog tracers helps to localize tumors and to evaluate the potential for chronic treatment of patients with inoperable SST receptor-positive tumors. By radioiodination of the tyrosine residue, a SST analog can be used for radiodiagnosis and therapy.

One method for using radiolabelled SST analogs is to label tyrosine containing analogs with iodine. For example, methods for using radiolabelled SST analogs that have been modified to contain a tyrosine amino acid were disclosed in Bomanji et al., (J. Nucl. Med., 33:1121, 1992). International patent application WO 96/39161 discloses multi-tyrosinated SST analogs in which the N-terminal of the peptides is extended with tyrosine residues, for radioiodination and subsequent diagnosis and treatment.

One application of radiolabelled SST analogs is radio-guided surgery. Surgical intervention can be optimized by intraoperative detection of tissue-bound (¹²⁵I-Tyr3)-Octreotide administered before operation. This technique has been successfully utilized in surgery of medullary thyroid cancer, carcinoids and islet cell tumors. High specific activity is achieved by the multi-tyrosinated SST analogs as a result of multiple sites for iodination provided by the additional tyrosines.

Another labeling method is reduction of a disulfide bridge, which provides two sulfhydryl groups for chelation with ^{99m}Tc (Kolan and Thakur Peptide Res. 9:144, 1996). Certain peptides can be labeled directly without a loss of functional specificity but others must be labelled using bifunctional chelating agents, which are covalently coupled to the analogs on one hand and form a complex with radiometals on the other hand.

Methods for labeling peptides with ^{99m}Tc are described in U.S. Pat. No. 5,716,596 and U.S. Pat. No. 5,620,675. Peptides are conjugated to chelators accepting different radioisotopes, i.e., ⁹⁹ mTc, ¹⁸⁶Re and ¹⁸⁸Re. U.S. Pat. No. 5,720,934 discloses details of a general method on radiolabelling ligands with ^{99m}Tc. A series of patents on radiolabelled SST analogs, describes cyclic (U.S. Pat. No. 5,932,189, WO 95/00553 and WO 96/04308) and linear (U.S. Pat. No. 5,620,675, WO 95/03330) peptides with 10-16 residues and high affinity for SST receptors. The cyclic peptides disclosed do not comprise a disulfide bond. An N₂S₂ type chelating ligand containing two nitrogen and two sulfur atoms for chelate formation, and use for cyclic and linear hexapeptide SST analogs, is disclosed in international applications WO 96/11954 and WO 96/11918. A disulfide-bridged SST analog with specific chelating groups is claimed in European application no. 714911. Analogs that contain at least 2 cysteine residues that form a disulfide or wherein the disulfide is reduced to the sulfhydryl form, are disclosed in U.S. Pat. No. 5,225,180. The compounds are stated to have improved tumor/kidney distribution ratios over conventional SST analogs, thus reducing kidney radiation exposure. International application WO 94/00489 and U.S. Pat. No. 5,871,711 disclose SST-derived peptide reagents for preparation of scintigraphic imaging agents. The SST analogs are labeled with Tc-99m, ¹⁸⁶Re and ¹⁸⁸Re through complexation.

U.S. Pat. No. 5,382,654 describes aminothiol ligands (N₂S₂ and N₃S) which can be conjugated to a SST analog peptide and can accommodate a metal ion, which can be a radiometal. For diagnostic purposes, ⁹⁹ mTc and ⁶²Cu are suggested for complex formation, while ¹⁸⁶Re, ⁶⁷Cu, ¹⁸⁸Re and ⁶⁰Co ions can be used for radiotherapy.

The effect of labeling methods and peptide sequence on ^{99m}Tc SST analogs was reviewed by Decristoforo C. and Mather S. J. (Eur. J. Nucl. Med., 26:869, 1999). It is concluded that the selection of the labeling approach as well as the right choice of the peptide structure are crucial for labeling peptides with ^{99m}Tc to achieve complexes with favorable activity and biodistribution. The authors further stated that further advantages due to different receptor specificity remain a topic for further investigations.

Recently, a number of ^{99m}Tc-labeled bioactive peptides have proven to be useful diagnostic imaging agents. Okarvi S. M. (Nuc. Med. Comm. 20:1093, 1999) reviews the recent developments in ^{99m}Tc-labeled peptide-based radiopharmaceuticals. Pearson et al. (J. Med. Chem., 39:1361, 1996) describe the chemistry and biology of ^{99m}Tc labeled SST analogs.

A radiolabelled SST analog, ¹¹¹In-DTPA-(D)Phe-Octreotide (OctreoScan, Mallinkrodt), has high diagnostic capacity for neuroendocrine tumors and lymphomas while its applicability for other tumors such as melanomas is lower. Labeled Octreotide analogs bind to SST-R2 and SST-R5. Scinitigraphy using this compound has become a valuable diagnostic tool to determine the extent of tumor disease and for planning surgical treatment (Ahlman et al., Br. J. Surg., 81: 1144, 1994). Octreotide labelled with ¹¹¹In has been shown to detect a variety of neuroendocrine tumors with high specificity and sensitivity and becomes a valuable tool in diagnosis, but it suffers form at least one major drawback: the cost. Vapreotide (RC-160) was labeled with ^{99m}Tc directly and also by using a bifunctional chelating agent (Thakur et al., Nucl. Med. Biol., 24:105, 1997). The radiolabelled SST analog was successfully evaluated in nude mice bearing experimental human prostate cancer. The compound ^{99m}Tc-Depreotide was successfully used in the evaluation of solitary pulmonary nodules in phase II/III clinical trial (Blum et al., Chest 117:1232, 2000). Recently (Aparici et al. Eur. J. Nuc. Med. 27:1754, 2000), SST receptor imaging has been used successfully (utilizing ¹¹¹In-pentetreotide) for detection of cardiac allograft rejection. Cardiac rejection process usually presents with lymphocyte infiltration, which indicates the severity of the rejection and the necessity of treatment. Activated lymphocytes express SST receptors thus SST receptor imaging could be used to target them. Somatostatin receptor imaging may predict impending rejection at least one week before the endomyocardial biopsy becomes positive and thus allow earlier intervention in the event of rejection.

A variety of radionuclides are known to be useful for radioimaging, including ⁶⁷Ga, ⁶⁸Ga, ^{99m}Tc, ¹¹¹In, ¹²³I or ¹²⁵I. The sensitivity of imaging methods using radioactively-labeled peptides is much higher than other techniques known in the art, since the specific binding of the radioactive peptide concentrates the radioactive signal over the cells of interest, for example, tumor cells. This is particularly important for endocrine-active gastrointestinal tumors, which are usually small, slow-growing and difficult to detect by conventional methods. Technetium-99m (^{99m}Tc, t_{1/2}=6 h, E_{γ}=140 keV) is the radionuclide of choice by virtue of its cost-effectiveness, availability and desirable nuclear characteristics. It is a decay product of ⁹⁹Mo. Because of its short half-life, it does not induce unnecessary radiation burden to a patient long after examinations are carried out, and its gamma ray energy is highly efficient for external imaging. ^{99m}Tc is used in over 90% of the diagnostic nuclear medicine procedures. Other radionuclides have effective half-lives, that are much longer (for example, ¹¹¹In, which has a half-life of 60-70 h), are toxic (for example, ¹²⁵I) or are expensive (¹¹¹In which is a cyclotron-produced radionuclide).

U.S. Pat. No. 4,980,147 discloses ^{99m}Tc compounds used as radiopharmaceutical imaging agents and particularly for conducting renal function imaging procedures. The preferred compound claimed is ^{99m}Tc-mercaptoacetyl-glycylglycylglycine (^{99m}Tc-MAG3). This and related compounds are used without conjugation with a SST or other peptide analog. U.S. Pat. No. 4,883,862 discloses the compound mercaptosuccinyl-glycylglycylglycine and its complexes with ^{99m}Tc for use as renal agents. The mercaptosuccinyl-glycylglycylglycine is made by coupling glycylglycylglycine with S-acetyl-mercapto succinic anhydride.

Other types of chelators that covalently connect the targeting biomolecule either directly or through a linker to the metallic radionuclide, which strongly coordinates to the chelator, are chelators having eight donor atoms that form stable five- to six-membered rings through metal complexation. Well known examples are DTPA (diethylenetriaminepentaacetic acid) and DOTA (1,4,7,10-tetraazacyclododecane-1,4,7,10-tetraacetic acid). DTPA complexes ¹¹¹In well and ¹¹¹In-DTPA is found in one of two commercially available SST-R radiopharmaceuticals, OctreoScan. DOTA forms stable complexes with many transition metal ions and because of this it has been deemed the universal chelator. It is an especially good complexing ligand for the lanthanide(III) ions, many of which are available as useful radionuclides. In a non-radionuclide application, natural Gd is used with two DOTA analogs as MRI contrast agents, gadoteridol and gadobutrol. The use of DOTA, DTPA and similar chelators for therapeutic somatostatin analogs (particularly lanthanide) radiopharmaceuticals was reviewed by Liu S and Edwards D S., (Bioconjug Chem 12,7-34, 2001).

Improved Peptide Analogs

As a result of major advances in organic chemistry and in molecular biology, many bioactive peptides can now be prepared in quantities sufficient for pharmacological and clinical use. Thus in the last few years new methods have been established for the treatment and diagnosis of illnesses in which peptides have been implicated.

However, the use of peptides as therapeutic and diagnostic agents is limited by the following factors: a) tissue penetration; b) low metabolic stability towards proteolysis in the gastrointestinal tract and in serum; c) poor absorption after oral ingestion, in particular due to their relatively high molecular mass or the lack of specific transport systems or both; d) rapid excretion through the liver and kidneys; and e) undesired side effects in non-target organ systems, since peptide receptors can be widely distributed in an organism.

It would be desirable to achieve peptide analogs with greater specificity thereby achieving enhanced clinical selectivity. It would be most beneficial to produce conformationally constrained peptide analogs overcoming the drawbacks of the native peptide molecules, thereby providing improved therapeutic properties.

A novel conceptual approach to the conformational constraint of peptides was introduced by Gilon, et al., (Biopolymers 31:745, 1991) who proposed backbone to backbone cyclization of peptides. The theoretical advantages of this strategy include the ability to effect cyclization via the carbons or nitrogens of the peptide backbone without interfering with side chains that may be crucial for interaction with the specific receptor of a given peptide. Further disclosures by Gilon and coworkers (WO 95/33765, WO 97/09344, U.S. Pat. No. 5,723,575, U.S. Pat. No. 5,811,392, U.S. Pat. No. 5,883,293 and U.S. Pat. No. 6,265,375), provided methods for producing building units required in the synthesis of backbone cyclized peptide analogs. The successful use of these methods to produce backbone cyclized peptide analogs of bradykinin analogs (U.S. Pat. No. 5,874,529), and backbone cyclized peptide analogs having somatostatin activity was also disclosed (WO 98/04583, WO 99/65508, U.S. Pat. No. 5,770,687 and U.S. Pat. No. 6,051,554). All of these methods are incorporated herein in their entirety, by reference.

There remains a need for synthetic SST analogs having increased in vivo stability, to be used therapeutically, as scintigraphic agents when radiolabelled with Tc-99m or other detectable radioisotopes for use in imaging tumors in vivo, and as radiotherapeutic agents when radiolabelled with a cytotoxic radioisotope such as rhenium-188. It would be desirable to achieve peptide analogs with greater specificity to receptor subtypes thereby achieving enhanced diagnostic selectivity to elucidate the specific SST receptor profile in each individual for planning further therapy and/or surgery. Backbone cyclized SST analogs that specifically fulfill these needs are provided by this invention.

None of the background art teaches or suggests the radiolabelled-backbone cyclized somatostatin analogs disclosed herein having improved diagnostic and therapeutic activity and selectivity.

### SUMMARY OF THE INVENTION

The present invention provides novel somatostatin analogs that are backbone cyclic peptide analogs for therapeutic and diagnostic applications, including radio-therapeutic and radio-diagnostic applications, in particular the present invention provides backbone cyclic SST analogs useful for scintigraphic imaging. The novel analogs according to the present invention having SST receptor subtype specific profiles, may be used for individualizing the diagnosis and treatment of tumors by application of receptor-specific reagents.

Distinct from native SST and SST analogs known in the art, the cyclic peptides of the present invention are backbone cyclized SST analogs which posses unique and superior properties such as chemical and metabolic stability, selectivity, increased bioavailability and improved pharmacokinetics. These analogs are further labeled with radioisotopes provided that the labeling methods and the radioisotopes maintain or increase the favorable properties of these backbone cyclic SST analogs.

According to the present invention, novel radiolabelled peptide analogs which are characterized in that they incorporate novel building units with bridging groups attached to the alpha nitrogens of alpha amino acids, are disclosed. Specifically, these compounds are backbone cyclized somatostatin analogs comprising a peptide sequence of three to twenty four amino acids, each analog incorporating at least one building unit, said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue. Preferably, the peptide sequence incorporates 3 to 14 residues, more preferably 4 to 12 amino acids, most preferably 5-9 amino acids.

The present invention provides for the first time the possibility of obtaining a panel of backbone cyclized radiolabelled analogs with specific somatostatin receptor selectivity or with combinations of receptor selectivity. That enables diagnostic and therapeutic uses in different types of cancers according to the specific needs of each patient and each disease.

According to the present invention it is now disclosed that preferred radiolabelled somatostatin analogs are octapeptide analogs with improved affinity and selectivity to specific somatostatin subtypes. Preferred analogs include novel backbone cyclic analogs of somatostatin which display receptor selectivity to SST-R subtypes 2 or to SST-R subtypes 2 and 5. More preferred radiolabelled somatostatin analogs may advantageously include bicyclic structures containing at least one cyclic structure connecting two building units and a second cyclic structure which is selected from the group consisting of side-chain to side-chain; backbone to backbone and backbone to terminal. Most preferred analogs are bicyclic structures containing one bridge connecting two building units and a second disulfide bridge. These bicyclic analogs are preferably selective to the subtype 2 somatostatin receptor.

Additional preferred analogs according to the present invention are backbone cyclic peptides of 3-5 amino acids. These analogs have an additional advantages related to their very low molecular weight of about 600 to 700 daltons, in comparison to the most common somatostatin synthetic analogs which usually are heptapeptides or octapeptides. Such low molecular weight is advantageous in terms of improved tissue (including tumor) penetration and lower production cost. These analogs are therefore particularly useful for diagnostic and therapeutic purposes.

The invention further provides backbone cyclic peptide reagents capable of being radiolabelled to form radiodiagnostic and radiotherapeutic agents, comprising a backbone cyclized somatostatin analog covalently linked to a radiolabel-binding moiety. In preferred embodiments according to the present invention the chelating moiety comprising four donor atoms. In more preferred analogs the chelating moiety comprises a moiety having eight donor atoms. According to the present invention the chelator can be linked to the analog via any free functional group available in the peptide. In most preferred analogs the chelator is covalently bound to the terminal nitrogen of the parent peptide. In certain preferred embodiments of these structures, two cysteine residues are used to chelate a radioisotope.

Preferred chelating moieties according to the present invention include those in which the four donor atoms are either three nitrogens and one sulfur (N3S) or two nitrogens and two sulfurs (N₂S₂) and, through metal complexation, stable 5- to 6-membered rings are formed according to the general Formula No. 1:

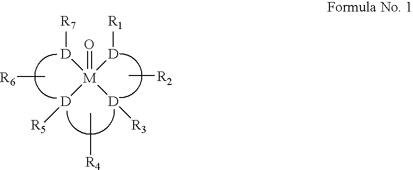

wherein the Ds represent the four donor atoms which are selected from N₃S and N₂S₂;

the half-circles represent two- or three-carbon bridges between the donor atoms;

the R groups are (i) single or multiple substitutions, and (ii) located on a position selected from the donor atoms and the carbon bridges; and M is a metal atom.

M is preferably selected from Re and Tc in the +5 oxidation state.

The R groups are preferably selected from the group of cyclic peptide, oxo, hydroxy, a hydrocarbon, hydrogen, a linking or spacing group connecting the cyclic peptide analog and the chelating moiety.

Chelators of the N₃S type, where a peptidyl N comprises the fourth donor atom, are for example: mercaptoacetyl-Gly-Gly- (MAG2), mercaptoethyl-Gly-Gly (MEG2). Chelators of the N₂S₂ type are for example constructs of: Cys-Gly-mercaptoacetyl (Cys-Gly-MA), Cys-Gly-mercaptoethyl (Cys-Gly-ME), diaminopropionic acid-mercaptoacetyl-mercaptoacetyl (Dpr-MA-MA), diaminopropionic acid-mercaptoacetyl-mercaptoethyl (Dpr-MA-ME), diaminopropionic acid-mercaptoethyl-mercaptoacetyl (Dpr-ME-MA), diaminopropionic acid-mercaptoethyl-mercaptoethyl (Dpr-ME-ME).

A more preferred embodiment of a radiolabel-binding moiety according to the present invention is: mercaptoacetyl-Gly-Gly-Gly- (MAG3).

Another more preferred embodiment of the above is MAG3-oxometal complex described in Formula No. 2.

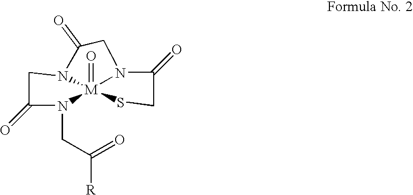

wherein the metal, M, is either Tc or Re in the +5 oxidation state;

the oxo group can be oriented either up or down;

and R represents the conjugated cyclic peptide analog.

Additional preferred embodiments comprise chelating moieties to form oxorhenium (V) or oxotechnetium (V) complexes having-1, neutral, +1, or +2 electronic charges.

Most preferred chelating moieties according to the present invention include chelators having eight donor atoms that, through metal complexation, form stable five- to six-membered rings. In one most preferred moiety three of the donor atoms are nitrogens and five are oxygens as described in Formula No. 3:

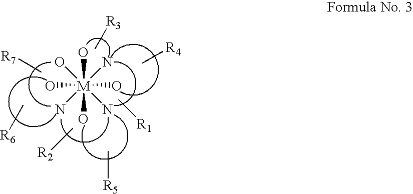

wherein the R groups are (i) single or multiple substitutions, and (ii) located on a position selected from one of the donor atoms or the carbon bridges;

the half-circles represent two- or three-carbon bridges between the donor atoms; and

M is an oxometal group.

M is preferably selected from (i) ReO and TcO with the metal in the +5 oxidation state and (ii) Indium in the +3 oxidation state.

the R groups are preferably selected from the group of cyclic peptide, oxo, hydroxy a hydrocarbon, hydrogen, or any linking or spacing group connecting the cyclic peptide analog and the chelating moiety.

In another most preferred chelating moiety four of the donor atoms are nitrogens and four are oxygens as described in Formula No. 4:

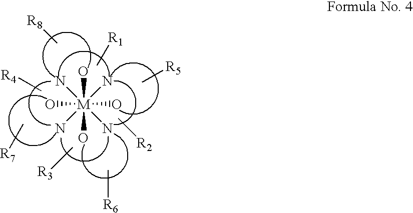

wherein the R groups are (i) single or multiple substitutions, and (ii) located on a position selected from one of the donor atoms or the carbon bridges;

the half-circles represent two- or three-carbon bridges between the donor atoms; and

M is an oxometal group.

M is preferably selected from the group of indium, yttrium, lutetium, gallium and gadolinium in the +3 oxidation state.

Thus, most preferred compounds according to the present invention comprise backbone cyclized somatostatin analogs comprising a chelator having eight donor atoms that, through metal complexation, form stable five- to six-membered rings. Most preferred chelator moieties are DTPA (diethylenetriaminepentaacetic acid) in which three of the donor atoms are nitrogens and five are oxygens and DOTA (1,4,7,10-tetraazacyclododecane-1,4,7,10-tetraacetic acid), In which four of the donor atoms are nitrogens and four are oxygens. Most preferred compounds comprising DTPA are further labeled with indium-111, additional preferred compounds are labeled with rhenium and a radionuclide of technetium. Most preferred compounds comprising DOTA are further labeled with radioisotopes of indium, yttrium, lutetium, gallium and gadolinium.

In other preferred analogs the peptide is coupled to the chelator via a linker to form a structure of the general Formula No. 5:

Z-Q-PTR Formula No. 5

wherein Z is a chelating moiety comprising: (i) four donor atoms selected form the group of N₃S and N₂S₂ that through metal complexation form three five- to six-membered rings or (ii) eight donor atoms that, through metal complexation, form stable five- to six-membered rings; Q is a direct bond or a linker moiety which can be coupled to a free functional group of the peptide; and PTR denotes a backbone cyclized SST analog according to the present invention.

Preferably, the linker Q is connected to the N-terminal of the peptide, more preferably the linker is selected from diaminopropionic acid (Dpr), diaminobutyric acid (Dab), aminohexanoic acid, polyethylene glycol (PEG), 4-aminobutyric acid, 6-aminocaproic acid, and β-alanine. Most preferably, Z is selected from the group of MAG3, DOTA or DTPA and Q is a direct bond.

Some of the preferred analogs according to the present invention may comprise two or more isomers. The present invention includes such isomers either in combination or individually isolated.

The invention provides radiolabelled backbone cyclic peptides that are scintigraphic imaging agents, radiodiagnostic agents and radiotherapeutic agents. Scintigraphic imaging agents of the invention comprise backbone cyclic peptide reagents radiolabelled with gamma-radiation emitting isotopes, preferably ^{99m}Tc for use in diagnostic imaging (single photon emission computed tomography, gamma camera, planar detector probes or devices for intraoperative use, positron emission tomography).

Radiotherapeutic agents of the invention comprise backbone cyclic peptide reagents radiolabelled with a cytotoxic radioisotope (having α or β emission). The most preferred cytotoxic radioisotopes according to the present invention are rhenium-186 and rhenium-188. Additional preferred radionuclides according to the invention are radioisotopes of indium, yttrium, lutetium, gallium and gadolinium. Combination embodiments, wherein a particular complex is useful both in scintigraphic imaging and in targeted radiotherapy, are also provided by the invention. Methods for making and using such backbone cyclic peptides, backbone cyclic reagents and radiolabelled embodiments thereof are also provided.

The currently most preferred backbone cyclized SST analogs according to the present invention are now disclosed.

One embodiment is a compound having the general Formula No. 6:

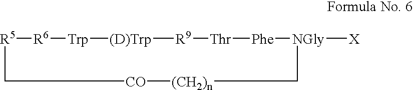

wherein n is 1 to 5;

X designates a terminal carboxy acid, amide or alcohol group;

R⁵ is 1,3-dicarbonyl-cyclopropane, phthalic acid, maleic acid, glutamic acid, valeric acid, diaminoethane-CO, diaminopropane-CO, or GABA;

R⁶ is Phe or Tyr; and

R⁹ is (L)- or (D)-Lys.

Preferably:

R⁶ is Phe;

R⁹ is Lys;

n is 3; and

X is an amide.

Alternative preferred analogs are tetra peptides having the general Formula No. 7:

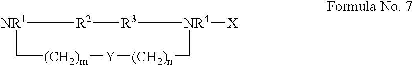

wherein m and n are 1 to 5;

X designates a terminal carboxy acid, amide or alcohol group;

R1 is Trp, (L)- or (D)-Lys, Ala, or Phe;

R2 is Ala, (L)- or (D)-Trp, or Lys;

R3 is (D)Trp, (L)- or (D)-Phe, Lys, Pro, or (D)Ala;

R4 is Lys, (D)Phe, (L)- or (D)-Ala, Trp, Gly; and

Y is amide, thioether, thioester, disulfide, urea, carbamate, or sulfonamide.

Another preferred analog according to the present invention is directly labeled with a radioisotope through chelation by two Cys side-chains, and has the following structure:

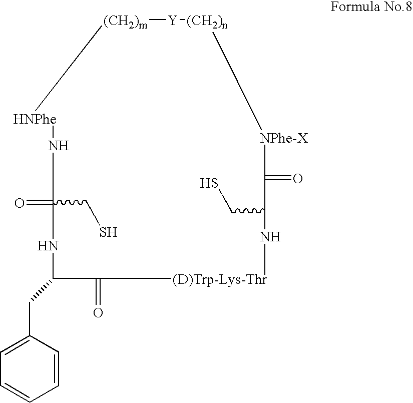

wherein m and n are 1 to 5;

X designates a terminal carboxy acid, amide or alcohol group;

Y is amide, thioether, thioester disulfide, urea, carbamate, or sulfonamide; and

the two cysteine residues are independently L or D.

the sulfur atoms of the cysteine residues together with neighboring nitrogen atoms chelate the oxometal, forming an N₂S₂ complex in which, including the metal, either two five-membered rings, one five membered ring and one six membered ring, or two six-membered rings are formed. The oxometal was left out of this drawing for clarity. The potential nitrogen donor atoms are indicated in boldface type. In each case, the oxo group has two possible orientations.

Another preferred embodiment is a compound of the general Formula No. 9:

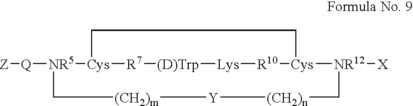

wherein m and n are 1 to 5;

X designates a terminal carboxy acid, amide or alcohol group;

Q is a direct bond or a linker moiety which can be coupled to any free functional group of the peptide;

Z is a chelating moiety comprising: (i) four donor atoms selected form the group of N₃S and

N₂S₂ that through metal complexation form 5- to 6-membered rings or (ii) eight donor atoms that, through metal complexation, form stable five- to six-membered rings;

R⁵ is (D)- or (L)-Phe or (D)- or (L)-Ala.

R⁷ is (D)- or (L)-Trp, (D)- or (L)-Phe, (D)- or (L)-1Nal, or (D)- or (L)-2Nal, or (D)- or (L) Tyr;

R10 is Thr, Gly, Abu, Ser, Cys, Val, (D)- or (L)-Ala, or (D)- or (L)-Phe;

R¹² is (D)- or (L)-Phe or (D)- or (L)-Ala; and

Y is amide, thioether, thioester disulfide, urea, carbamate, or sulfonamide.

The most preferred analog according to Formula No. 9 (designated herein as PTR 3261) is a compound wherein:

X is amide;

Q is a direct bond;

Z is MAG3;

R⁵ is Phe;

R⁷ is Phe;

R¹⁰ is Thr;

R¹² is Phe;

Y is amide;

m is 3; and

n is 3.

PTR 3261 having the formula MAG3-PheC3-Cys-Phe-(D)Trp-Lys-Thr-Cys-PheN3-NH2, is a bicyclic analog of SST in which one bridge connects the two building units (PheC3 and PheN3) and the second is a disulfide bridge formed between the two cysteine residues. The chelating moiety MAG3 is covalently linked to the N-terminal of the analog. This analog is used as an intermediate for synthesizing the compounds designated herein below PTR 3265 and PTR 3271.

Additional preferred analogs according to Formula No. 9 are selected from the group of:

MA-Dap(MA)-Gly-Phe(C3**)-Cys*-Phe-(D)Trp-Lys-Thr-Cys*-Phe(N3**)-NH₂ denoted PTR3303;

MA-Dap(Gly)-Gly-Phe(C3**)-Cys*-Phe-(D)Trp-Lys-Thr-Cys*-Phe(N3**)-NH₂ denoted PTR3305;

Gly-Dap(MA)-Gly-Phe(C3**)-Cys*-Phe-(D)Trp-Lys-Thr-Cys*-Phe(N3**)-NH₂ denoted PTR3307;

MA-Dap(MA)-GABA-Phe(C3**)-Cys*-Phe-(D)Trp-Lys-Thr-Cys*-Phe(N3**)-NH₂ denoted PTR3309;

MA-Dap(Gly)-GABA-Phe(C3**)-Cys*-Phe-(D)Trp-Lys-Thr-Cys*-Phe(N3**)-NH₂ denoted PTR3311;

Gly-Dap(MA)-GABA-Phe(C3**)-Cys*-Phe-(D)Trp-Lys-Thr-Cys*-Phe(N3**)-NH₂ denoted PTR3313.

wherein one asterisk denotes that the two form a disulfide bridge, and two asterisks denotes that a second bridging group is connected between the N^{α}-ω-functionalized derivative of marked residues.

Additional more preferred analogs are compounds the general Formula No. 10:

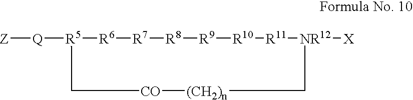

wherein n is 1 to 5;

X designates a terminal carboxy acid, amide or alcohol group;

Z is a chelating moiety comprising: (i) four donor atoms selected form the group of N₃S and N₂S₂ that, through metal complexation, form three 5- to 6-membered rings or (ii) eight donor atoms that, through metal complexation, form stable five- to six-membered rings;

Q is a direct bond or a linker moiety which can be coupled to a free functional group of the peptide;

R⁵ is diaminobutyric acid or diaminopropionic acid.

R⁶ is (D)- or (L)-Phe or Tyr;

R⁷ is (D)- or (L)-Trp, (D)- or (L)-Phe, (D)- or (L)-1Nal or (D)- or (L)-2Nal, or Tyr;

R⁸ is (D)- or (L)-Trp;

R⁹ is (D)- or (L)-Lys;

R¹⁰ is Thr, Gly, Abu, Ser, Cys, Val, (D)- or (L)-Ala, or (D)- or (L)-Phe;

R¹¹ is (D)- or (L)-Phe, (D)- or (L)-Ala, Nle, or Cys; and

R¹² is Gly, Val, Leu, (D)- or (L)-Phe or 1Nal or 2Nal.

Preferably:

X is amide;

Q is a direct bond;

Z is MAG3;

R⁵ is Dab;

R⁶ is Phe;

R⁷ is Trp;

R⁸ is (D)Trp;

R⁹ is Lys;

R¹⁰ is Thr;

R¹¹ is Phe;

R¹² is Gly; and

n is 3.

This preferred analog has the following structure:

MAG3-Dab*-Phe-Trp-(D)Trp-Lys-Thr-Phe-GlyC3-X

wherein X is amide, and the asterisk denotes that the bridging group is connected between the N^{α}-ω-functionalized derivative of the Gly residue (GlyC3) and a free amine of the Dab residue, and the MAG3 is connected to the other free amine of the Dab residue.

More preferably,

X is amide;

Q is selected from the group of a direct bond, Gly, β-Ala and GABA;

Z is DOTA or DTPA;

R⁵ is Dab;

R⁶ is Phe;

R⁷ isTrp;

R⁸ is (D)Trp;

R⁹ is Lys;

R¹⁰ is Thr;

R¹¹ is Phe;

R¹² is Gly; and

n is 3.

More preferred analogs labeled with indium, according to Formula No. 10 are the compounds:

In-DTPA-Gly-Dab*-Phe-Trp-(D)Trp-Lys-Thr-Phe-GlyC3*-NH₂ denoted PTR 3319;

In-DTPA-Dab*-Phe-Trp-(D)Trp-Lys-Thr-Phe-GlyC3*-NH₂ denoted PTR 3337;

In-DTPA-P-Ala-Dab*-Phe-Trp-(D)Trp-Lys-Thr-Phe-GlyC3*-NH₂ denoted PTR 3339;

In-DTPA-GABA-Dab*-Phe-Trp-(D)Trp-Lys-Thr-Phe-GlyC3*-NH₂ denoted PTR 3341;

In-DTPA-5-aminopentanoic acid-Dab*-Phe-Trp-(D)Trp-Lys-Thr-Phe-GlyC3*-NH₂ denoted PTR 3343;

In-DTPA-3-aminomethylbenzoic acid-Dab*-Phe-Trp-(D)Trp-Lys-Thr-Phe-GlyC3*-NH₂ denoted PTR 3345.

Additional preferred analogs, labeled with rhenium, based on formula no. 10 are the compounds:

ReO-MA-Dpr(MA)-Dab*-Phe-Trp-(D)Trp-Lys-Thr-Phe-GlyC3*-NH₂;

ReO-MA-Dpr(MA)-Gly-Dab*-Phe-Trp-(D)Trp-Lys-Thr-Phe-GlyC3*-NH₂;

ReO-MA-Dpr(MA)-βAla-Dab*-Phe-Trp-(D)Trp-Lys-Thr-Phe-GlyC3*-NH₂;

ReO-MA-Dpr(MA)-GABA-Dab*-Phe-Trp-(D)Trp-Lys-Thr-Phe-GlyC3*-NH₂;

ReO-MA-Dpr(MA)-5-aminopentanoic acid-Dab*-Phe-Trp-(D)Trp-Lys-Thr-Phe-GlyC3*-NH₂;

ReO-MA-Dpr(MA)-6-aminohexanoic acid-Dab*-Phe-Trp-(D)Trp-Lys-Thr-Phe-GlyC3*-NH₂;

ReO-MA-Gly-Gly-Gly-Dab*-Phe-Trp-(D)Trp-Lys-Thr-Phe-GlyC3*-NH₂;

ReO-MA-Gly-Gly-Gly-Gly-Dab*-Phe-Trp-(D)Trp-Lys-Thr-Phe-GlyC3*-NH2;

ReO-MA-Gly-Gly-Gly-GABA-Dab*-Phe-Trp-(D)Trp-Lys-Thr-Phe-GlyC3*-NH₂;

ReO-MA-Gly-Gly-Gly-5-aminopentanoic acid-Dab*-Phe-Trp-(D)Trp-Lys-Thr-Phe-GlyC3*-NH₂;

The most preferred radiolabelled analogs of Formula No. 8 are the compounds:

ReO-MA-Dpr(Gly)-Dab*-Phe-Trp-(D)Trp-Lys-Thr-Phe-GlyC3*-NH₂ denoted PTR 3395, and the metal free analog MA-Dpr(Gly)-Dab*-Phe-Trp-(D)Trp-Lys-Thr-Phe-GlyC3*-NH₂ denoted PTR 3361;

ReO-MA-Dpr(Gly)-Gly-Dab*-Phe-Trp-(D)Trp-Lys-Thr-Phe-GlyC3*-NH₂ and the metal free analog MA-Dpr(Gly)-Gly-Dab*-Phe-Trp-(D)Trp-Lys-Thr-Phe-GlyC3*-NH₂ denoted PTR 3397;

ReO-MA-Dpr(Gly)-PAla-Dab*-Phe-Trp-(D)Trp-Lys-Thr-Phe-GlyC3*-NH₂ denoted PTR 3399, and the metal free analog MA-Dpr(Gly)-PAa-Dab*-Phe-Trp-(D)Trp-Lys-Thr-Phe-GlyC3*-NH₂ denoted PTR 3359;

ReO-MA-Gly-Gly-Gly-6-aminohexanoic acid-Dab*-Phe-Trp-(D)Trp-Lys-Thr-Phe-GlyC3*-NH₂ denoted PTR 3401, and the metal free analog MA-Gly-Gly-Gly-6-aminohexanoic acid-Dab*-Phe-Trp-(D)Trp-Lys-Thr-Phe-GlyC3*-NH₂ denoted PTR 3357.

These analogs were found to exhibit high affinity (in the nanomolar to subnanomolar range) for human and rat SST-R2 as well as selectivity (up to 1000 fold) toward rat SST-R2 versus the other rat SST-R subtypes.

A currently preferred analog is designated herein as PTR 3265 which is PTR 3261 labeled with rhenium [oxorhenium(V)-PTR 3261]. PTR 3265 has improved affinity of binding to the SST-R subtype 2. The analog contains two isomers (3265A and 3265B) and is described in the following Formula No. 11:

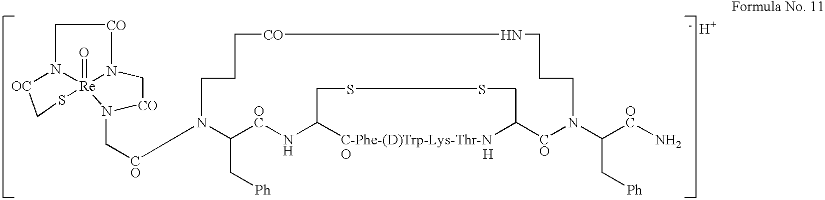

Another more preferred analog is PTR 3271 which is PTR 3261 labeled with technetium (oxo[^{99m}Tc]technetium(V)-PTR 3261). The analog, which contains two isomers (3271A and 3271B), is described in the following formula No. 12:

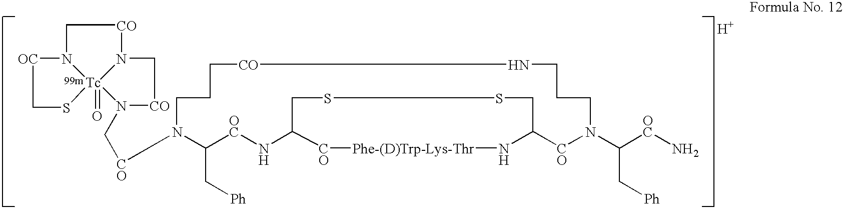

These backbone cyclized SST peptide analogs are prepared by incorporating at least one N^{α}-ω-functionalized derivative of an amino acid into a peptide sequence and subsequently selectively cyclizing the functional group with one of the side chains of the amino acids in the peptide sequence or with another co-functionalized amino acid derivative.

It is an advantage of the most preferred analogs according to the invention that the non-disulfide backbone cyclic linkage is stable in the presence of reducing agents used to form a complex between a radioisotope and a radiolabel binding moiety of the invention comprising a thiol group. The disulfide linkage, when present in the bicyclic peptides, is also stable to reduction by virtue of protection from reduction by the second backbone cyclization. The SST analogs of the invention are thus advantageous over linear SST and previously known cyclic disulfide-bridged SST analogs.

It is another advantage of the SST analogs provided by this invention that the cyclic covalent linkage acts to protect the peptide from degradation by exopeptidases. Further, the bicyclic structure confers a high degree of conformational rigidity to the peptide that can act to enhance binding of the peptide to its biological target (i.e., the SST receptor).

Backbone cyclized analogs of the present invention may be used as diagnostic compositions in methods for diagnosing cancer and imaging the existence of tumors or their metastases, and in detection of allograf rejection including but not limited to cardiac allograft rejection. The methods for diagnosis of cancer and allograft rejection comprise administering to a mammal, including a human patient, a backbone cyclic analog or analogs labeled with a detectable tracer which is selected from the group consisting of a radioactive isotope and a non-radioactive tracer. The methods for the diagnosis or imaging of cancer and allograft rejection using such compositions represent another embodiment of the invention.

The pharmaceutical compositions comprising pharmacologically active radiolabelled backbone cyclized SST agonists or antagonists and a pharmaceutically acceptable carrier or diluent represent another embodiment of the invention, as do the methods for the treatment of cancers in targeted radiotherapy using such compositions. The pharmaceutical compositions according to the present invention advantageously comprise at least one backbone cyclized peptide analog which is selective for one or more SST receptor subtypes. These pharmaceutical compositions may be administered by any suitable route of administration, including orally, topically or systemically. Preferred modes of administration include but are not limited to parenteral routes such as intravenous and intramuscular injections, as well as via intra-nasal administration or oral ingestion.

The invention further provides a method for treating or diagnosing somatostatin-related diseases in animals, preferably humans, comprising administering a therapeutically effective amount of backbone cyclic SST analogs of the invention. In some preferred embodiments, the reagent is radioactively labeled with ¹⁸⁶Re or ¹⁸⁸Re. In other preferred embodiments the backbone cyclic analog is not labeled.

Another aspect of the present invention provides methods for preparing radiotherapeutic and radiodiagnostic radiopharmaceuticals, including preferably scintigraphic imaging agents. Each such reagent comprises a backbone cyclized SST analog covalently linked to a radiolabel-binding moiety. The invention further provides kits for making and radiolabelling such compositions.

### BRIEF DESCRIPTION OF THE DRAWINGS

FIG. 1 describes the HPLC chromatogram of the crude material of PTR 3265 as described in example 3.

FIG. 2 describes the mass spectral analysis of PTR 3265A and PTR 3265B.

FIG. 3 demonstrates the competitive binding curve of PTR 3265A and 3265B tested on cloned human SST receptor type 2. These curves were used to calculate the IC₅₀ of the two compounds as described in example 4.

FIG. 4 depicts the high affinity of selected backbone cyclized compounds to human SST-R2 as described in example 8.

FIG. 5 depicts the selectivity of selected compounds to rat SST receptor subtype 2 as described in example 8.

### DETAILED DESCRIPTION OF THE INVENTION

According to the present invention, peptide analogs are cyclized via bridging groups attached to the alpha nitrogens of amino acids that permit novel non-peptidic linkages. In general, the procedures utilized to construct such peptide analogs from their building units rely on the known principles of peptide synthesis; most conveniently, the procedures can be performed according to the known principles of solid phase peptide synthesis.

The methods for design and synthesis of backbone cyclized analogs according to the present invention are disclosed in U.S. Pat. Nos. 5,811,392; 5,874,529; 5,883,293; 6,051,554; 6,117,974; 6,265,375, and international applications WO 95/33765; WO 97/09344; WO 98/04583; WO 99/31121; WO 99/65508; WO 00/02898; and WO 00/65467. All of these methods are incorporated herein in their entirety, by reference.

The most striking advantages of backbone cyclization are:

1) cyclization of the peptide sequence is achieved without compromising any of the side chains of the peptide thereby decreasing the chances of sacrificing functional groups essential for biological recognition (e.g. binding to specific receptors), and function.

2) optimization of the peptide conformation is achieved by allowing permutation of the bridge length, and bond type (e.g., amide, disulfide, thioether, thioester, urea, carbamate, or sulfonamide, etc.), bond direction, and bond position in the ring.

3) when applied to cyclization of linear peptides of known activity, the bridge can be designed in such a way as to minimize interaction with the active region of the peptide and its cognate receptor. This decreases the chances of the cyclization arm interfering with recognition and function, and also creates a site suitable for attachment of tags such as radioactive tracers, cytotoxic drugs, light active substances, or any other desired label.

Distinct from native SST and SST analogs known in the background art, the cyclic peptides of the present invention are backbone cyclized SST analogs which possess unique and superior properties such as chemical and metabolic stability, selectivity, increased bioavailability and improved pharmacokinetics. These analogs are further labeled with radioisotopes provided that the labeling methods and the radioisotopes maintain or increase the favorable properties of these backbone cyclic SST analogs.

Terminology and Definitions

The term “antagonist of somatostatin” in the context of the present invention preferably means that these molecules are able to reduce or prevent at least one of the actions of somatostatin.

The term “agonist of somatostatin” preferably means that the molecules are capable of mimicking at least one of the actions of somatostatin.

The term “linker” denotes a chemical moiety whose purpose is to link, covalently, a chelating moiety and a backbone cyclic peptide. The linker may be also used as a spacer whose purpose is to allow distance between the chelating moiety (thus the radiometal) and the backbone cyclic peptide.

The term “chelating agent” as used herein denotes a chemical moiety whose purpose is to stably form a chelating agent (or chelator)-metal complex. The complex is formed through electron donation from certain electron-rich atoms on the chelating agent to the electron-poor metal. The chelating agent typically has four donor atoms. The preferred donor atom for oxorhenium(V) and oxotechnetium(V) is nitrogen and the most preferred donor atom is sulfur.

The term “scintigraphic imaging agent” as used herein is meant to encompass a radiolabelled agent capable of being detected with a radioactivity detecting means (including but not limited to a planar camera, a gamma-camera, a single photon emission (computed) tomography (SPECT or SPET) or any hand-held probe (e.g. Geiger-Muller counter or a scintillation detector) or device for use intraoperatively or otherwise in the detection of tumors.

As used herein “peptide” indicates a sequence of amino acids linked by peptide bonds. The peptides according to the present invention comprise a sequence of 3 to 50 amino acid residues, preferably 3 to 24 residues, more preferably 5 to 18 amino acids. A peptide analog according to the present invention may optionally comprise at least one bond which is an amide-replacement bond such as urea bond, carbamate bond, sulfonamide bond, hydrazine bond, or any other covalent bond.

The term “analog” further indicates a molecule which has the amino acid sequence according to the invention except for one or more amino acid changes. The design of appropriate “analogs” may be computer assisted.

Whenever “peptide of the invention” or “analogs of the invention” are mentioned in the present specification and claims, also salts and functional derivatives thereof are contemplated, as long as the biological activity of the peptide with respect to SST is maintained. Functional derivatives of the peptides of the invention covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties. Salts of the peptides of the invention contemplated by the invention are physiologically acceptable organic and inorganic salts.

As used herein the term “backbone cyclic peptide” or “backbone cyclic analog” denote an analog of a linear peptide which comprising a peptide sequence of preferably 3 to 24 amino acids that incorporates at least one building unit, said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue. More preferably, the peptide sequence incorporates 3-14 amino acids, still more preferably it incorporates 4-12 amino acids, and most preferably 5-9 amino acids.

A “building unit” indicates an N^{α} derivatized a amino acid of the general Formula No. 13:

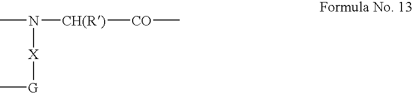

wherein X is a spacer group selected from the group consisting of alkylene, substituted alkylene, arylene, cycloalkylene and substituted cycloalkylene; R′ is an amino acid side chain, optionally bound with a specific protecting group; and G is a functional group selected from the group consisting of amines, thiols, alcohols, carboxylic acids, sulfonates and esters, and alkyl halides; which is incorporated into the peptide sequence and subsequently selectively cyclized via the functional group G with one of the side chains of the amino-acids in said peptide sequence or with another co-functionalized amino acid derivative.

The methodology for producing the building units is described in international patent applications published as WO 95/33765 and WO 98/04583 and in U.S. Pat. Nos. 5,770,687 and 5,883,293 all of which are expressly incorporated herein by reference thereto as if set forth herein in their entirety.

The building units are abbreviated by the three letter code of the corresponding modified amino acid followed by the type of reactive group (N for amine, C for carboxyl), and an indication of the number of spacing methylene groups. For example, GlvC2 describes a modified Gly residue with a carboxyl reactive group and a two carbon methylene spacer, and PheN3 designates a modified phenylalanine group with an amino reactive group and a three carbon methylene spacer. In generic formulae the building units are abbreviated as R with a superscript corresponding to the position in the sequence preceded by the letter N, as an indication that the backbone nitrogen at that position is the attachment point of the bridging group specified in said formulae.

The compounds herein disclosed may have asymmetric centers. All chiral, diastereomeric, and racemic forms are included in the present invention. Many geometric isomers of double bonds and the like can also be present in the compounds disclosed herein, and all such stable isomers are contemplated in the present invention.

By “stable compound” or “stable structure” is meant herein a compound that is sufficiently robust to survive isolation to a useful degree of purity from a reaction mixture, and formulation into an efficacious therapeutic agent.

The term, “substituted” as used herein and in the claims, means that any one or more hydrogen atoms on the designated atom is replaced with a selection from the indicated group, provided that the designated atom's normal valency is not exceeded, and that the substitution results in a stable compound.

When any variable (for example R, X, Z, etc.) occurs more than one time in any constituent or in any Formula herein, its definition on each occurrence is independent of its definition at every other occurrence. Also, combinations of substituents and/or variables are permissible only if such combinations result in stable compounds.

As used herein and in the claims, the phrase “therapeutically effective amount” means that amount of novel backbone cyclized peptide analog or composition comprising same to administer to a host to achieve the desired results for the indications disclosed herein, such as but not limited to cancer, endocrine disorders, inflammatory diseases, and gastrointestinal disorders.

Certain abbreviations are used herein to describe this invention and the manner of making and using it. For instance, ACN refers to acetonitrile, AcOH refers to acetic acid, Alloc refers to allyloxycarbonyl, Boc refers to the t-butyloxycarbonyl, CPM refers to counts per minute, DCM refers to dichloromethane, DIEA refers to diisopropyl-ethyl amine, DMF refers to dimethyl formamide, EDT refers to ethanedithiol, DOTA refers to 1,4,7,10-tetraazacyclododecane-1,4,7,10-tetraacetic acid. DTPA refers to diethylenetriaminepentaacetic acid, Fmoc refers to fluorenylmethoxycarbonyl, HOBT refers to 1-hydroxybenzotriazole, HPLC refers to high pressure liquid chromatography, GABA refers to gamma aminobutyric acid; MAG2 refers to mercaptoacetyl-Gly-Gly, MAG3 refers to mercaptoacetyl-Gly-Gly-Gly, MA refers to mercaptoacetate, ME refers to mercaptoethyl, MEG refers to mercaptethyl-Gly-, MEG2 refers to mercaptoethyl-Gly-Gly-, mCi refers to millicurie, MS refers to mass spectrometry, NMM refers to N-methylmorpholine, NMP refers to 1-methyl-2-pyrolidonone, PEG refers to polyethylene glycol, PET refers to positron emission tomography, PyBOP refers to benzotriazole-1-yl-oxy-tris-pyrrolidino-phosphonium hexafluorophosphate, PyBrOP refers to bromo-tris-pyrrolidino-phosphonium hexafluorophosphate, RT refers to room temperature, SPECT refers to single photon emission computed tomography, SPET refers to single photon emission tomography, SRIF refers to Somatotropin Release Inhibitory Factor, SST refers to somatostatin, SST-R refers to somatostatin receptor, tBu refers to the tertiary butyl, TFA refers to trifluoroacetic acid, THF refers to tetrahydrofuran, TIS refers to triisopropylsilane.

The amino acids used in this invention are those which are available commercially or are available by routine synthetic methods. Certain residues may require special methods for incorporation into the peptide, and either sequential, divergent and convergent synthetic approaches to the peptide sequence are useful in this invention. Natural coded amino acids and their derivatives are represented by three-letter codes according to IUPAC conventions. When there is no indication, the L isomer was used. The D isomers are indicated by “(D)” before the residue abbreviation. List of Non-coded amino acids: Abu refers to 2-aminobutyric acid, Dab refers to diaminobutyric acid, Dpr and Dap both refer to diaminopropionic acid, GABA refers to gamma aminobutyric acid, 1Nal refers to 1-naphthylalanine, 2Nal refers to 2-naphtylalanine, and Nle refers to norleucine.

Conservative substitution of amino acids as known to those skilled in the art are within the scope of the present invention. Conservative amino acid substitutions includes replacement of one amino acid with another having the same type of functional group or side chain e.g. aliphatic, aromatic, positively charged, negatively charged.

Preferred Embodiments

According to the present invention, novel peptide analogs which are characterized in that they incorporate novel building units with bridging groups attached to the alpha nitrogens of alpha amino acids, are disclosed. Specifically, these compounds are backbone cyclized somatostatin analogs comprising a peptide sequence of three to twenty four amino acids, each analog incorporating at least one building unit, said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue. Preferably, the peptide sequence incorporates 3 to 14 residues, more preferably 4 to 12 amino acids, most preferably 5-9 amino acids.

Backbone cyclic analogs of the present invention bind with high affinity to a defined subset of the human SST receptors. This receptor selectivity indicates the potential physiological selectivity in vivo. Furthermore, the present invention provides for the first time the possibility to obtain a panel of backbone cyclized radiolabelled analogs with specific SST receptor selectivity or with combinations of receptor selectivity. This enables diagnostic and therapeutic uses in different types of cancers according to the specific needs of each patient and each disease.

According to the present invention it is now disclosed that preferred SST analogs are octapeptide analogs with improved affinity and selectivity to specific SST subtypes. Preferred analogs include novel backbone cyclic analogs of SST which display receptor selectivity to SST-R subtypes 2 or to SST-R subtypes 2 and 5. More preferred SST analogs may advantageously include bicyclic structures containing at least one cyclic structure connecting two building units and a second cyclic structure which is selected from the group consisting of side-chain to side-chain; backbone to backbone and backbone to terminal. Most preferred analogs are bicyclic structures containing one bridge connecting two building units and a second disulfide bridge. These bicyclic analogs are preferably selective to the subtype 2 SST receptor.

Additional preferred analogs according to the present invention are backbone cyclic peptides of 3-5 amino acids. Some of these preferred analogs were obtained using a pharmacophore based search algorithm. These analogs have an additional advantages related to their very low molecular weight of about 600 to 700 daltons in comparison to the most common somatostatin synthetic analogs which usually are heptapeptides or octapeptides. Such low molecular weight is advantageous in terms of improved tissue (including tumor) penetration and lower production cost.

The invention further provides backbone cyclic peptide reagents capable of being radiolabelled to form radiodiagnostic and radiotherapeutic agents, each comprising a backbone cyclized SST analog covalently linked to a radiolabel-binding chelating moiety. In preferred embodiments according to the present invention the chelating moiety comprising four donor atoms. According to the present invention the chelator can be linked to the analog in any free functional group available at the peptide. In most preferred analogs the chelator is covalently bound to the terminal nitrogen of the parent peptide. In some preferred embodiments of these structures, two cysteine residues are used to chelate a radioisotope.

Some prior art compounds contain a non-protected disulfide bond, that is unstable in the presence of reducing agents useful in the production of radiolabelled complexes with various radiolabel binding moieties. The biological activity of the previously-known reagents was thereby reduced, and the efficacy of scintigraphic imaging agents derived therefrom compromised, by reduction of these peptide structure-determining disulfide bonds. Such destabilization of the structure of the backbone cyclic analogs of the present invention does not occur due to the lack of such non-protected unstable disulfide bonds in the SST receptor binding compounds comprising the present invention. Thus, the use of thiol-containing radiolabel chelating moieties and the use of reducing agents to form radioisotope complexes therewith does not result in loss of biological activity of the SST receptor binding compounds of this invention.

Preferred chelating moieties according to the present invention include those in which the four donor atoms are either three nitrogens and one sulfur (N3S) or two nitrogens and two sulfurs (N₂S₂) and, through metal complexation, stable 5- to 6-membered rings are formed according to the general Formula No. 1:

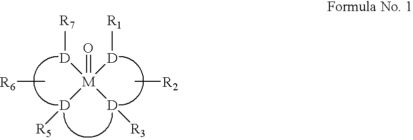

wherein the Ds represent the four donor atoms which are selected from N₃S and N₂S₂;

the half-circles represent two- or three-carbon bridges between the donor atoms;

the R groups are (i) single or multiple substitutions, and (ii) located on a position selected from the donor atoms and the carbon bridges; and M is a metal atom.

M is preferably selected from Re and Tc in the +5 oxidation state.

The R groups are preferably selected from the group of cyclic peptide, oxo, hydroxy, a hydrocarbon, hydrogen, a linking or spacing group connecting the cyclic peptide analog and the chelating moiety.

Chelators of the N₃S type, where a peptidyl N comprises the fourth donor atom, are for example: mercaptoacetyl-Gly-Gly- (MAG2), mercaptoethyl-Gly-Gly (MEG2). Chelators of the N₂S₂ type are for example constructs-of: Cys-Gly-mercaptoacetyl (Cys-Gly-MA), Cys-Gly-mercaptoethyl (Cys-Gly-ME), diaminopropionic acid-mercaptoacetyl-mercaptoacetyl (Dpr-MA-MA), diaminopropionic acid-mercaptoacetyl-mercaptoethyl (Dpr-MA-ME), diaminopropionic acid-mercaptoethyl-mercaptoacetyl (Dpr-ME-MA), diaminopropionic acid-mercaptoethyl-mercaptoethyl (Dpr-ME-ME).

A more preferred chelating moiety of the above is MAG3-oxometal complex described in Formula No. 2.

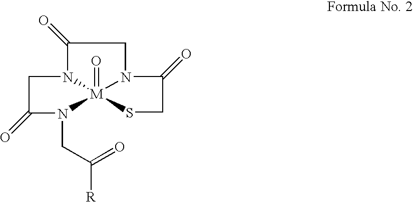

wherein the metal, M, is either Tc or Re in the +5 oxidation state;

the oxo group can be oriented either up or down;

and R represents the conjugated cyclic peptide analog.

Chelators of the N₃S type, where a peptidyl N comprises the fourth donor atom, are, e.g.: mercaptoacetyl-Gly-Gly- (MAG2-), mercaptoacetaldehyde-Gly-Gly (MALDG2-). Chelators of the N₂S₂ type are for example constructs of: Cys-Gly-MA, Cys-Gly-MALD, Dpr-MA-MA, Dpr-MA-MALD, Dpr-MALD-MA, Dpr-MALD-MALD.

The preferred embodiment of a radiolabel-binding moiety according to the present invention is: mercaptoacetyl-Gly-Gly-Gly- (MAG3-), which forms a stable square pyramidal complex with oxorhenium(V) or oxotechnetium(V) having a charge of-1. Additional preferred embodiments comprise chelating moieties to form oxorhenium(V) or oxotechnetium(V) complexes having −1, neutral, +1, or +2 electronic charges as described in the following table:

TABLE NO 1

| | | Oxo-metal(V) |
|---|---|---|
| | Donor chemical descriptor | charge when complexed |
| N₃S | amide-amide-amide-sulfhydryl | −1 |
| | amide-amide-amine-sulfhydryl | Neutral |
| | amide-amine-amine-sulfhydryl | +1 |
| | amine-amine-amine-sulfhydryl | +2 |
| N₂S₂ | Amide-amide-sulfhydryl-sulfhydryl | −1 |
| | Amide-amine-sulfhydryl-sulfhydryl | Neutral |
| | Amine-amine-sulfhydryl-sulfhydryl | +1 |

Most preferred chelating moieties according to the present invention include chelators having eight donor atoms that through metal complexation form stable five- to six-membered rings. In one most preferred moiety three of the donor atoms are nitrogens and five are oxygens as described in Formula No.3:

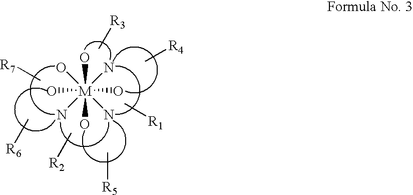

wherein the R groups are (i) single or multiple substitutions, and (ii) located on a position selected from one of the donor atoms or the carbon bridges;

the half-circles represent two- or three-carbon bridges between the donor atoms; and

M is an oxometal group.

M is preferably selected from (i) ReO and TcO with the metal in the +5 oxidation state and (ii) Indium in the +3 oxidation state.

the R groups are preferably selected from the group of cyclic peptide, oxo, hydroxy, a hydrocarbon, hydrogen, or any linking or spacing group connecting the cyclic peptide analog and the chelating moiety.

In another most preferred chelating moiety four of the donor atoms are nitrogens and four are oxygens as described in Formula No. 4:

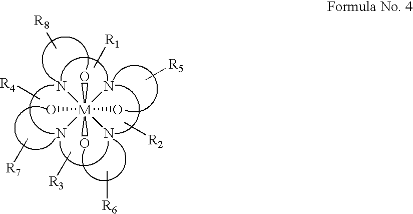

wherein the R groups are (i) single or multiple substitutions, and (ii) located on a position selected from one of the donor atoms or the carbon bridges;

the half-circles represent two- or three-carbon bridges between the donor atoms; and

M is an oxometal group.

M is preferably selected from the group of indium, yttrium, lutetium, gallium and gadolinium in the +3 oxidation state.

In other preferred analogs the peptide is coupled to the chelator via a linker to form a structure of the general Formula No. 5:

Z-Q-PTR Formula No. 5

wherein Z is a chelating moiety comprising: (i) four donor atoms selected form the group of N₃S and N₂S₂ that through metal complexation form three five- to six-membered rings or (ii) eight donor atoms that, through metal complexation, form stable five- to six-membered rings; Q is a direct bond or a linker moiety which can be coupled to a free functional group of the peptide; and PTR denotes a backbone cyclized SST analog according to the present invention.

Preferably, the linker Q is connected to the N-terminal of the peptide, more preferably the linker is selected from diaminopropionic acid (Dpr), diaminobutyric acid (Dab), aminohexanoic acid, PEG, 4-aminobutyric acid, 6-aminocaproic acid, and β-alanine. Most preferably, Z is selected from the group of MAG3. DOTA or DTPA and Q is a direct bond.

The invention provides radiolabelled backbone cyclic peptides that are scintigraphic imaging agents, radiodiagnostic agents and radiotherapeutic agents. Scintigraphic imaging agents of the invention comprise backbone cyclic peptide reagents radiolabelled with gamma-radiation emitting isotopes, preferably ^{99m}Tc for use in diagnostic imaging (single photon emission computed tomography, gamma camera, planar, detector probes or devices for intraoperative use). Any other technetium or rhenium radioisotopes having decay characteristics making them useful in radionuclide imaging (including positron emission tomography, PET), capable of complexation with the backbone cyclic analogs of the invention, are also encompassed by the present invention.

Radiotherapeutic agents of the invention comprise backbone cyclic peptide reagents radiolabelled with a cytotoxic radioisotope (α or β emission). Most preferred cytotoxic radioisotopes according to the present invention are rhenium-186 and rhenium-188. Combination embodiments, wherein such a complex is useful both in scintigraphic imaging and in targeted radiotherapy, are also provided by the invention. Any other technetium or rhenium radioisotopes having decay characteristics making them useful in radiotherapy, capable of complexation with the backbone cyclic analogs of the invention, are also are also encompassed by the present invention.

Somatostatin is a tetradecapeptide hormone whose numerous regulatory functions are mediated by a family of five receptors, whose expression is tissue dependent. Receptor specific analogs of SST are believed to be valuable diagnostic and therapeutic agents in the treatment and diagnosis of various diseases. Attempts to design small peptide analogs having this selectivity have not been highly successful. It has now unexpectedly been found that the conformationally constrained backbone cyclized SST analogs of the present invention, are highly selective to SST receptor subtypes and are therefore useful for diagnosis and treatment of conditions where specific SST receptors are expressed in specific tissues. Such conditions are preferably different types of cancers such as colon cancer, growth hormone-secreting pituitary adenoma, thyroid cancer, gastric carcinoid, small cell lung carcinoma, melanoma, medullary non-Hodgkin's lymphoma, and breast cancer and other types of cancer. In addition, the backbone cyclized SST analogs of the present invention may be used for detection of allograft rejection including but not limited to cardiac allograft rejection.

Backbone cyclized analogs of the present invention may be used as diagnostic compositions in methods for diagnosing cancer and imaging the existence of tumors or their metastases, and in detection of allograft rejection including but not limited to cardiac allograft rejection. The methods for diagnosis of cancer and allograft rejection comprise administering to a mammal, including a human patient, a backbone cyclic analog or analogs labeled with a detectable tracer which is selected from the group consisting of a radioactive isotope and a non-radioactive tracer. The methods for the diagnosis or imaging of cancer and allograft rejection using such compositions represent another embodiment of the invention.

The imaging agents provided by the invention have utility for tumor imaging, particularly for imaging primary and metastatic neoplastic sites wherein said neoplastic cells express SST receptors, and in particular such primary and especially metastatic tumor cells that have been clinically difficult to detect and characterize using conventional methodologies. The imaging reagents according to the present invention may be used for visualizing organs, and tumors, in particular gastrointestinal tumors, myelomas, small cell lung carcinoma and other APUDomas, endocrine tumors such as medullary thyroid carcinomas and pituitary tumors, brain tumors such as meningiomas and astrocytomas, and tumors of the prostate, breast, colon, and ovaries can also be imaged.

The ^{99m}Tc labeled diagnostic reagents are preferably administered intravenously in a single unit injectable dose. These reagents may be administered in any conventional medium for intravenous injection such as an aqueous saline medium. Generally, the unit dose to be administered has radioactivity of about 1 to 30 mCi. The solution to be injected at unit dosage is from about 0.1 to about 10 mL. After intravenous administration, imaging in vivo can be performed any time from immediately up to and including four physical decay half lives following administration. Any method of scintigraphic imaging such as gamma scintigraphy, can be utilized in accordance with the present invention.

Radioactively-labeled scintigraphic imaging agents according to the present invention are provided having radioactivity in solution containing at concentrations of from about 1 mCi to 100 mCi per mL.

The pharmaceutical compositions comprising pharmacologically active backbone cyclized SST agonists or antagonists and a pharmaceutically acceptable carrier or diluent represent another embodiment of the invention, as do the methods for the treatment of cancers in targeted radiotherapy using such compositions. The pharmaceutical compositions according to the present invention advantageously comprise at least one backbone cyclized peptide analog which is selective for one or two SST receptor subtypes. These pharmaceutical compositions may be administered by any suitable route of administration, including orally, topically or systemically. Preferred modes of administration include but are not limited to parenteral routes such as intravenous and intramuscular injections, as well as via intra-nasal administration or oral ingestion. The preferred doses for administration of such pharmaceutical compositions range from about 0.1 μg/kg to about 20 mg/kg body weight/day.

The pharmaceutical compositions may preferably be used to promote regression of certain types of tumors, particularly those that express SST receptors. Furthermore, the pharmaceutical compositions can also be used to reduce the hormonal hypersecretion that often accompanies certain cancers, such as the APUDomas. Other conditions of which the compounds of the present invention are useful for treatment are endocrine disorders, gastrointestinal disorders, diabetes-associated complications, pancreatitis, autoimmune diseases, and inflammatory diseases, allograft rejection, atherosclerosis and restenosis.

The invention further provides a method for alleviating somatostatin-related diseases in animals, preferably humans, comprising administering a therapeutically effective amount of backbone cyclic SST analogs of the invention to the animal. In some preferred embodiments the backbone cyclic analog is unlabeled.

In some preferred embodiments, rhenium-186 or rhenium-188 may be used for radiotherapy of certain tumors if the reagent is radioactively labeled with cytotoxic radioisotopes such as ¹⁸⁶Re or ⁸⁸Re. In preferred embodiments, the amount of the SST analog administered is from about 0.1 μg/kg to about 20 mg/kg body weight/day. For this purpose, an amount of radioactive isotope from about 10 mCi to about 200 mCi may be administered via any suitable clinical route, preferably by intravenous injection.

Another aspect of the present invention provides methods for preparing radiotherapeutic and radiodiagnostic radiopharmaceuticals, preferably scintigraphic imaging agents, and the reagents required to make them. Each such reagent is comprised of a backbone cyclized SST analog covalently linked to a radiometal complexing moiety. For example, scintigraphic imaging agents provided by the invention comprise ^{99m}Tc labeled complexes formed by reacting the reagents of the invention with ^{99m}Tc in the presence of an agent capable of reducing [^{99m}Tc]pertechnetate ion (+7 metal oxidation state, that elutes from the ⁹⁹Mo/^{99m}Tc generator found commonly in the nuclear medicine clinic or nuclear pharmacy) to the oxo[⁹9 mTc]technetium species (+5 metal oxidation state). Preferred reducing agents include but are not limited to dithionite, stannous and ferrous ions. Such ^{99m}Tc complexes of the invention are also formed by labeling the peptide analogs of the invention with 99 mTc by ligand exchange of a prereduced ^{99m}Tc complex. In this case, a weak chelator is present in the in situ reduction cocktail, but the reagents of this invention are not initially present. The reagents of this invention are then added to the solution containing the +5 oxidation state oxo[^{99m}Tc]technetium “weak chelator” complex, forming the more stable oxo[^{99m}Tc]technetium complex with the reagents of this invention.

The invention further provides kits for radiolabelling backbone cyclic SST analogs. In a preferred embodiment of the invention, a kit for preparing [^{99m}Tc]technetium-labeled peptide analogs is provided. An appropriate amount of the backbone cyclic analog is introduced into a vial containing a reducing agent, such as stannous chloride, in an amount sufficient to label the analog with ^{99m}Tc. An appropriate amount of a transfer ligand (a weak oxo[^{99m}Tc]technetium chelator such as tartrate, citrate, gluconate, 2,5-dihydroxybenzoate, glucoheptanoate or mannitol, for example) can also be included. The kit may also contain additives such as salts to adjust the osmotic pressure, buffers to adjust the pH or preservatives to allow longer storage of either the cold kid or the final diagnostic radiopharmaceutical. The components of the kit may be in liquid, frozen or in dry form. In a preferred embodiment, the kit components are provided in lyophilized form.

Technetium-99m labeled imaging reagents according to the present invention may be prepared by the addition of an appropriate amount of ^{99m}Tc or ^{99m}Tc-complex into the vial containing the reagents according to the present invention, and reaction under appropriate conditions. Kits for preparing radiotherapeutic agents wherein the preferred radioisotopes are rhenium-186 and rhenium-188 are also provided.

Most Preferred Embodiments

The most preferred backbone cyclized SST analogs according to the present invention are compounds according to Formulae 5-12 defined herein above.

PTR 3261 having the formula MAG3-PheC3-Cys-Phe-(D)Trp-Lys-Thr-Cys-PheN3-NH₂, is a bicyclic analog of SST in which one bridge connects the two building units (Phe-C3 and Phe-N3) and the second is a disulfide bridge formed between the two cysteine residues. The chelating moiety MAG3 is covalentlv linked to the N-terminal of the analog. This analog is used as intermediate for synthesizing PTR 3265 and PTR 3271.

One currently preferred analog is PTR 3265 which is PTR 3261 labeled with rhenium [oxorhenium(V)-PTR 3261]. PTR 3265 has improved affinity of binding to the SST-R subtype 2 over that of its parent PTR 3261. The analog contains two isomers (3265A and 3265B) as described in the following scheme No. 1:

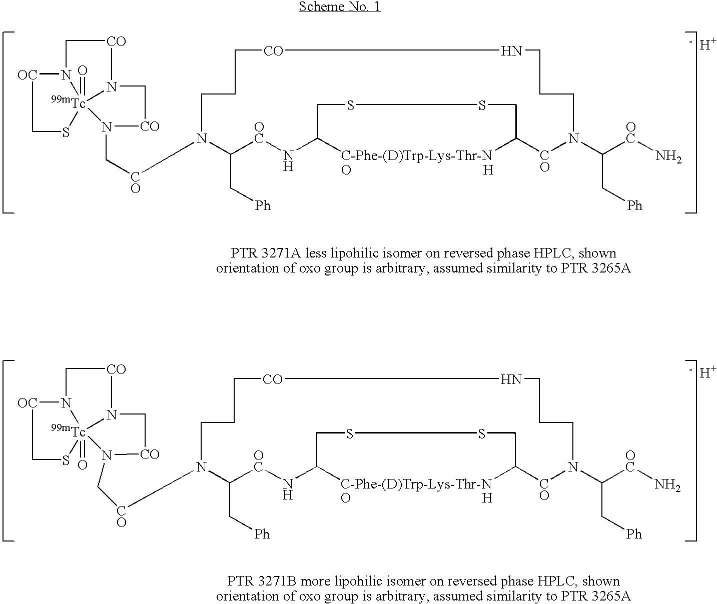

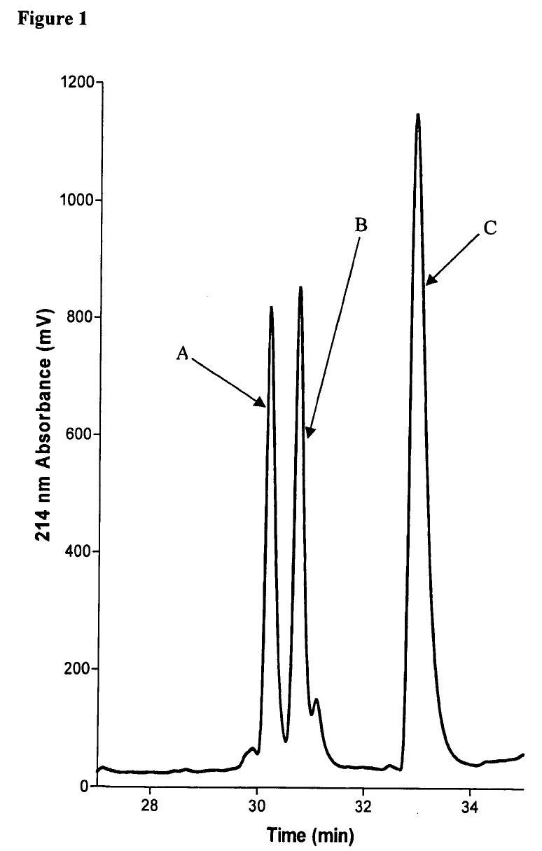

PTR 3265 comprises two isomers A and B. The existence of the two isomers was detected by existence of two peaks in the HPLC separation chromatogram (FIG. 1). The compounds of the two HPLC chromatogram peaks have the same molecular weight. The two isomers probably differ in the orientation of the rhenium(V)oxide. It is expected that the analog with technetium instead of rhenium will also be composed of two or more corresponding isomers. The present invention includes such isomers either in combination or individually isolated.

Another more preferred analog is PTR 3271 which is PTR 3261 labeled with technetium (oxo[^{99m}Tc]technetium(V)-PTR 3261). The analog contains two isomers (3271A and 3271B) as described in the following Scheme No. 2:

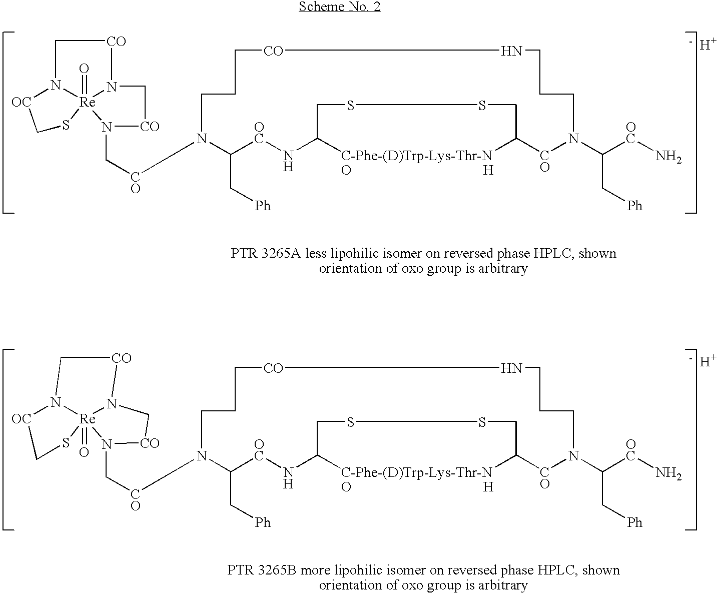

These backbone cyclized SST peptide analogs are prepared by incorporating at least one N^{α}-ω-functionalized derivative of an amino acids into a peptide sequence and subsequently selectively cyclizing the functional group with one of the side chains of the amino acids in the peptide sequence or with another co-functionalized amino acid derivative.

The backbone cyclic peptides of this invention are novel selective analogs and preferably bind with higher affinity to a single receptor of the SST receptor family. PTR 3265 is a bicyclic compound in which one bridge connects the two building units and the second is a disulfide bridge formed between two Cys residues. This analog is selective for SST receptor 2 and thus it is a potential candidate for treating malignancies expressing this receptor subtype without influencing other SST receptor activities, and after proper derivatization and labeling, for imaging. Similarly, other analogs are selective to SST receptor 5 and are thus candidates for imaging the therapy of malignancies expressing this receptor subtype. Radiolabelled derivatives of PTR 3173, such as PTR 3395, 3397 (when ReO-complexed), 3399 and 3401 are expected, like their parent, to bind both SST-R2 and SST-R5 and therefore may be used to detect and treat malignancies expressing both receptor types. PTR 3399 indeed binds both SST-R2 and SST-R5. Table No. 2 describes the affinity of some of the preferred analogs of the present invention to the SST-Rs in comparison to SRIF-14. Table No. 2: Concentration (nM) of SST analogs to inhibit SRIF binding to each human cloned SST receptors by 50%.

| | | | SST-R | | | | |
|---|---|---|---|---|---|---|---|
| PTR | | Sequence | 1 | 2 | 3 | 4 | 5 |
| SRIF- | | H-Ala¹-Gly²-Cys³-Lys⁴-Asn⁵-Phe⁶-Phe⁷-Trp⁸- | 2.8 | 0.23 | 1.2 | ND | 2.6 |
| 14 | | Lys⁹-Thr¹⁰-Phe¹¹-Thr¹²-Ser¹³-Cys¹⁴-OH | | | | | |
| 3205 | | PheC3-Cys-Phe-(D)Trp-Lys-Thr-Cys-PheN3- | 349 | 1.5 | 323 | 123 | 186 |
| | | NH₂ | | | | | |
| 3261 | | MAG3-PheC3-Cys-Phe-(D)Trp-Lys-Thr-Cys- | ND | 100 | ND | ND | ND |
| | | PheN3-NH₂ | | | | | |
| 3265A | | oxo[^{99m}Tc]technetium(V)-PTR 3261 | ND | 0.85 | ND | ND | ND |
| 3265B | | oxo[^{99m}Tc]technetium(V)-PTR 3261 | ND | 2.16 | ND | ND | ND |
| 3395 | 30012-02 | ReO-MA-Dpr(Gly)-Dab*-Phe-Trp-(D)Trp- | ND | 1.14 | ND | ND | ND |
| | | Lys-Thr-Phe-GlyC3*-NH₂ | | | | | |
| ReO- | 30012-05 | ReO-MA-Dpr(Gly)-Gly-Dab*-Phe-Trp- | 89 | 0.25 | 76 | 3.3 | 2.2 |
| 3397# | | (D)Trp-Lys-Thr-Phe-GlyC3*-NH₂ | | | | | |
| 3399# | 30012-08 | ReO-MA-Dpr(Gly)-βAla-Dab*-Phe-Trp- | 156 | 1.6 | 103 | 15 | 1.3 |
| | | (D)Trp- Lys-Thr-Phe-GlyC3*-NH₂ | | | | | |
| 3401# | 30012-24 | ReO-MA-Gly-Gly-Gly-6-aminohexanoic | 402 | 0.44 | 110 | 20 | 3.7 |
| | | acid-Dab* -Phe-Trp-(D)Trp-Lys-Thr-Phe- | | | | | |
| | | GlyC3*-NH₂ | | | | | |

ND Not determined.
The asterik denotes that the bridging group is connected between the free functional group of that residue the N^{α}-ω-functionalized derivative of the Gly residue.
#Determined in rat somatostatin receptors, all the other binding measurements were determined in human receptors.

The affinity of the preferred analogs PTR 3265A and PTR 3265B to type 2 SST receptor is in the subnanomolar-nanomolar range which makes these analogs potentially effective diagnostic and therapeutic compositions.

A 100-fold increase in potency (in affinity to the SST-R2) was observed in the chemical change from the non-complexed PTR 3261 to oxo-rhenium(V)-complexed PTR 3265A and 3265B. The phenomenon may be ascribed to the fact that the metal-complexed entity is chemically dissimilar to the non-complexed entity and thus has different electronic and torsional properties which affect the spatial positioning of the key pharmacophore moieties. Alternatively, considering the pharmacophore rigidity induced upon double cyclization, the phenomenon may be due to the fact that, for PTR 3261 versus PTR 3265, the MAG3 moiety occupies a larger chemical space when non-complexed than when metal-complexed. The larger chemical space occupied with the free MAG3 moiety would then be assumed to interfere with receptor-ligand binding.

It is an advantage of the most preferred analogs according to the invention that the non-disulfide backbone cyclic linkage is stable in the presence of reducing agents used to form a complex between a radioisotope and a radiolabel binding moiety of the invention comprising a thiol group. The disulfide linkage, when present in the bicyclic peptides, is also stable to reduction by virtue of protection from reduction by the second, backbone cyclization. The SST analogs of the invention are thus advantageous over linear SST and previously known cyclic disulfide-bridged SST analogs. The background art compounds contain a non-protected disulfide bond that is unstable in the presence of reducing agents useful in the production of radiolabelled complexes with various radiolabel binding moieties. The biological activity of the previously-known reagents was thereby reduced, and the efficacy of scintigraphic imaging agents derived therefrom compromised, by reduction of these peptide structure-determining disulfide bonds. Such destabilization of the structure of the backbone cyclic analogs of the present invention does not occur due to the lack of such non-protected unstable disulfide bonds in the SST receptor binding compounds comprising the present invention. Thus, the use of thiol-containing radiolabel chelating moieties and the use of reducing agents to form radioisotope complexes therewith does not result in loss of biological activity of the SST receptor binding compounds of this invention.

It is another advantage of the SST analogs provided by this invention that the cyclic covalent linkage acts to protect the peptide from degradation by exopeptidases. Further, the bicyclic structure confers a high degree of conformational rigidity to the peptide that can act to enhance binding of the peptide to its biological target (i.e., the SST receptor).

General Method for Synthesis, Purification and Characterization of Backbone Cyclic Peptides

Synthesis:

Resin: 1 g Rink amide or Tenta-gel resin, with loading of 0.2-0.7 mmol/g.

Fmoc-deprotection: With 7 mL of 20% piperidine in NMP (twice for 15 minutes). Followed by 5 washes with 10 mL NMP for 2 minutes with shaking.

Couplings:

1. Regular couplings (coupling to simple amino acids): with a solution containing 3 equivalents amino acid, 3 equivalents PyBroP and 6 equivalents of DIEA in 7 mL NMP. For 0.5-2 hours with shaking. Coupling is monitored by ninhydrin test and repeated until the ninhydrin solution remains yellow.

2. Coupling of His and Asn with a solution containing 5 equivalents DIC and 5 equivalents HOBT in 10 mL DMF.

3. Coupling to Gly building units: with a solution containing 3 equivalents amino acid, 3 equivalents PyBroP and 6 equivalents DIEA in 7 mL NMP. Twice for 1-4 hours with shaking.

4. Coupling to building units other than Gly: with a solution containing 5 equivalents amino acid, 1.5 equivalents triphosgene and 13 equivalents collidine in 15 mL dioxane or THF. Twice for 0.5-2 hours at 50° C. with shaking.

Removal of the Allyl and Alloc protecting groups of the building units: with 0.6 equivalent per Allyl or Alloc of Pd(PPh₃)₄ in 30 mL DCM containing 5% acetic acid and 2.5% NMM. For 1-4 hours with shaking.

Cyclization: with a solution containing 3 equivalents PyBOP and 6 equivalents DIEA in 7 mL NMP. For 0.5-2 hours with shaking. Cyclization is monitored by ninhydrin test and repeated if necessary.

Cleavage: with 82%-95% TFA supplemented with scavengers: 1-15% H₂₀, 1-5% TIS and O-5% EDT.

Purification:

An individual purification method for each backbone cyclic peptide is developed on analytical HPLC to optimize isolation of the cyclic peptide from other components. The analytical method is usually performed using a C-18 Vydac column 250×4.6 mm as the stationary phase and water/ACN containing 0.1% TFA mixture gradient. The preparative method is designed by adapting the analytical separation method to the preparative C-18 Vydac column. During the purification process, the peak containing the cyclic peptide is collected using a semi-automated fraction collector. The collected fractions are injected to the analytical HPLC to check purity. The pure fractions are combined and lyophilized.

Characterization:

The combined pure lyophilized material is analyzed for purity by HPLC, MS and capillary electrophoresis and by amino acid analysis for peptide content and amino acid ratio determination.

General Methods for Radiolabelling with Technetium

In forming a complex of radioactive technetium with the reagents of this invention, the ⁹⁹Mo/^{99m}Tc generator eluent, preferably containing sodium [^{99m}Tc]pertechnetate (+7 oxidation state), is reacted with the reagent in the presence of a reducing agent. The preferred reducing agent is stannous chloride, which reliably reduces Tc^{VII} to Tcv. Means for preparing such complexes are conveniently provided in a kit form comprising a sealed vial containing a predetermined quantity of a reagent of the invention to be labeled and a sufficient amount of reducing agent to label the reagent with Tc-99m. Alternatively, the complex may be formed by reacting a reagent of this invention with a pre-formed labile complex of technetium and another compound known as a transfer ligand. This process is known as ligand exchange and is well known to those skilled in the art. The labile complex may be formed using such transfer ligands as tartrate, citrate, gluconate, 2,5-dihydroxybenzoate, glucoheptanoate or mannitol, for example.

Indium Complexation on DTPA-Peptide Conjugate

10 mM citric acid (pH 2.5) and 10 mM trisodium citrate (pH 8) solutions are combined in the ratio 1:3 acid-sodium salt to give pH 6 10 mM citrate buffer. 10 mg crude DTPA-peptide conjugate is dissolved in 4 mL citrate buffer plus 5 mL acetonitrile. Indium chloride (1.4 mg) is added in 1.2 mL citrate buffer. The amount of indium added is equimolar to the intentionally overestimated peptide amount (assuming 100 percent of the crude product is DTPA-peptide conjugate). Mixing is continued at room temperature for 21-24 hours after which the peptide solution is sampled for analysis and lyophilized. Purification proceeds as usual.

Radioactive Indium-111 Complexation on DTPA-Peptide Conjugate

Indium-111 chloride in 0.02 N HCl (also containing ferric chloride) is added to a lyophylate of DTPA-peptide conjugate, gentisic acid, trisodium citrate, citric acid and inositol. After mixing, the mixture is allowed to stand at room temperature for at least 30 minutes for completion of the reaction.

General Method for Forming Metal Complexes with Crude Chelator-Cyclic Peptide Conjugates:

Crude chelator-cyclic peptide conjugates can be complexed with either oxorhenium(V) (tetradentate chelators, i.e., N₂S₂ and N₃S donors) or indium(III) (octadentate chelators, i.e., N₃O₅ donors). The post-cleavage crude is weighed and the molar amount is calculated, assuming the mass is 100% desired conjugate. Alternatively, the molar amount of conjugate is calculated based on the solid phase resin loading. The appropriate metal reagent is added at an equimolar amount. This strategy works with rhenium when the crude peptide is relatively pure (such as in the case of PTR-3173 derivatives), but not when the crude is impure (such as in the case of PTR-3205 derivatives). The strategy works with indium regardless of the level of purity of the crude material. By avoiding a chromatographic purification step, time and resources are saved.

General Method for In Vitro Screening of Somatostatin Analogs

The ability of the SST analogs of the invention to bind to SST receptors in vitro was demonstrated by assaying the ability of such analogs to inhibit binding of a radiolabelled SST analog to SST receptor-containing cell membranes.

The SST analogs were tested for their potency in inhibition of the binding of ¹²⁵I-Tyr¹¹-SRIF (based on the method described by Raynor et. al., Molecular Pharmacology 43: 838, 1993) to membrane preparations expressing the transmembranal SST receptors (SST-R1, 2, 3, 4 or 5). The receptor preparations used for these tests were either from the cloned human receptors selectively and stably expressed in Chinese Hamster Ovary (CHO) cells or from cell lines naturally expressing the SST-Rs. Typically, cell membranes were homogenized in Tris buffer in the presence of protease inhibitors and incubated for 30-40 minutes with ¹²⁵I-Tyr¹¹-SRIF with different concentrations of the tested sample. The binding reactions were filtered, the filters were washed and the bound radioactivity was counted in β counter after addition of scintillation solution. Non specific binding was defined as the radioactivity remaining bound in the presence of 1 μM unlabeled SRIF-14.

In Vivo Models for Evaluating the Activity of Somatostatin Analogs

The radiolabelled compounds of the present invention are tested in vivo for tumor uptake in xenografts derived from cell lines such as the following:

a. Rat pituitary adenoma cells (GH3) in nude rats.

b. Human colon adenocarcinoma cells (HT-29) in nude mice or nude rats.

c. Rat pancreatic acinar carcinoma cells (CA20948) in normal rats.

d. Rat pancreatic cancer cells (AR42J) in nude mice.

e. Human small cell lung carcinoma cells (NCI-H69) in nude mice.

f. Human pancreatic carcinoid cells (BON-1) in nude mice or nude rats.

g. LCC-18 cells in nude mice or nude rats.

Briefly, the cells are implanted intramuscularly in a suspension of 0.05 to 0.1 mL/animal, the tumors are allowed to grow to approximately 0.5 to 2 g, harvested, and used to implant a second, naive set of animals. Passaging in this fashion is repeated to generate successive generations of tumor-bearing animals. Third- to fifth-passage of tumor-bearing animals are injected intravenously with labeled compound. At selected times, the animals are sacrificed and harvested tissue samples are weighed and counted, along with an aliquot of the injected dose, in a gamma well-counter.

General In Vivo Imaging Methods

In vivo imaging of SST receptors expressed by animal tumor cells is performed essentially as described by Bakker et al. (1991, Life Sciences 49:1593-1601). Additional in vivo screening methods are described in details in Examples 6.

Conformationally constrained SST analogs constructed based in part on the sequences of a number of known biologically active peptides or based on previously unknown novel sequences are presented in the examples below. The following examples are intended to illustrate how to make and use the compounds and methods of this invention and are in no way to be construed as a limitation.

### EXAMPLES

The invention will now be illustrated in a non-limitative manner by the following Examples:

### Example 1

### Detailed Procedure of PTR 3261 Synthesis

Rink amide MBHA resin (1 g, 0.55 mmol) was swelled in NMP (4 h) in a glass reactor equipped with a sintered glass bottom. Fmoc was removed from the resin using two 15-min piperidine-NMP (1:3, 8 mL) treatments. After washing the resin thoroughly (NMP, 7×, 8 mL, 2 min), Fmoc-PheN3(Alloc)-OH (0.872 g, 1.65 mmol) was coupled in NMP (8 mL) with PyBroP (0.769 g, 1.65 mmol) and DIEA (0.560 mL, 3.30 mmol) as activating agents (1 h, RT). Following coupling, the modified resin was washed (NMP, 5×, 8 mL, 2 min). Reaction completion was checked by a qualitative ninhydrin (Kaiser) test. Fmoc removal and subsequent washing was carried out as described above, followed by washing with THF (3×, 8 mL, 2 min). Fmoc-Cys(Acm)-OH (1.14 g, 2.75 mmol) was coupled to the modified resin using triphosgene (0.269 g, 0.908 mmol) and collidine (1.02 mL, 7.70 mmol) in THF (15 mL) at 50° C. for 75 min. The Fmoc-Cys(Acm)-OH coupling procedure was repeated. Successive addition of Thr(tBu), Lys(Boc), (D)Trp(Boc), Phe, Cys(Acm) and PheC3(Allyl) was accomplished using standard coupling cycles as described above for Fmoc-PheN3(Alloc)-OH. Fmoc removal and amino acid addition were confirmed with ninhydrin tests. After washing the peptidyl resin with DCM (3×, 8 mL, 2 min) and DCM-AcOH-NMM (37:2:1, sparged with argon, 3×, 8 mL, 2 min), Allyl/Alloc deprotection was achieved by adding Pd(P(Ph)₃)₄ (1.5 g, 1.30 mmol) in DCM-AcOH-NMM (same as above, 40 mL) to the reactor, degassing by bubbling argon through the reactor's sintered glass bottom and then shaking vigorously for 2 h in the dark. The cyclic peptidyl resin was washed sequentially with (8 mL and 2 min per wash) DCM-AcOH-NMM (3×), CHCl₃ (5×) and NMP (3×). To eliminate residual Pd through complexation, washing was performed with an NMP solution of sodium diethyldithiocarbamic acid trihydrate (0.5% W/V, 2×, 8 mL, 2 min). Following three NMP washes, backbone cyclization was accomplished using PyBOP (0.858 g, 1.65 mmol) and DIEA (0.56 mL, 3.30 mmol) in NMP (10 mL) for 1 h and then again over night (same conditions). The peptidyl resin was washed with NMP, DMF and DMF-water (4:1) each three times for 2 min. Iodine (1.40 g, 5.52 mmol) in DMF-water (14 mL, 4:1) was added to the reactor which was shaken for 40 min to afford Acm deprotection and Cys-Cys cyclization. The peptidyl resin was washed extensively with DMF/water, DMF, NMP, DCM, CHCl₃ and 2% ascorbic acid in DMF. Approximately 75% (0.41 mmol) of the peptidyl resin was used for elaboration of the doubly cyclic peptide with the MAG3 moiety. Afler Fmoc deprotection and NMP washing as above, Fmoc-Gly-OH (0.610 g, 2.05 mmol) was coupled in DCM-DMF-NMP (6 mL) using PyBroP (0.956 g, 2.05 mmol) as activating agent and DIEA (0.724 mL, 4.1 mmol) as base. The procedure was repeated. Two additional Gly residues were attached using the normal Fmoc removal and PyBroP/DIEA coupling procedure described above. After removal of Fmoc from the third Gly residue and thorough washing (NMP, THF), S-tritylmercaptoacetic acid (0.693 g, 2.07 mmol) was coupled in THF (6 mL) using triphosgene (0.201 g, 0.678 mmol) as activating agent and collidine (0.765 mL, 5.78 mmol) as base (50° C., 75 min).

The peptidyl resin was washed with DCM and MeOH and dried first with air (30 min) and then under vacuum (30 min). The peptide was cleaved from the resin (with simultaneous trityl, tBu and Fmoc protecting group removal), under argon using TFA-TIS-water (38:1:1, 12 mL) first at 0° C. (15 min) and then at RT (75 min). The solution was filtered into a polypropylene tube and the resin was washed into the tube with cleavage cocktail and TFA. Concentration under a N₂ stream gave an oily residue (0.631 g) which was triturated to a brown solid with cold Et₂O. Drying under vacuum over 3 days gave crude PTR 3261 (191 mg). Reversed phase, preparative HPLC yielded 12.2 mg (8.41 μmol) pure PTR 3261 (yield 2.1% from resin).

### Example 2

### Reaction of PTR 3261 with Rhenium to Yield PTR 3265A & PTR 3265B

PTR 3261 (12.2 mg, 8.41 μmol) and trichlorooxobis(triphenylphosphine)rhenium(V) (8.03 mg, 9.64 μmol) were each dissolved in 6 mL DMF, cooled on ice and combined under argon. Stirring was continued on ice for 4 h after which the reaction was left to stir at room temperature for 2.5 days. Removal of DMF was achieved by first vacuum on a rotary evaporator (sample at 40° C.) and then lyophilization in water-ACN-0.1% TFA to give 19.8 mg crude.

### Example 3

### Characterization of PTR 3265A & PTR 3265B

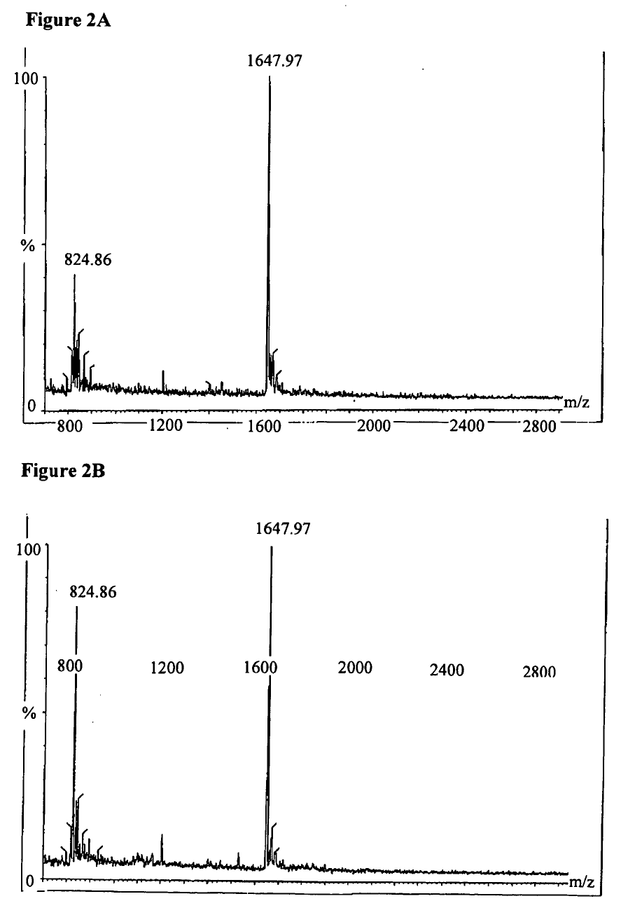

The crude material of PTR 3265 was analyzed by reversed phase HPLC on a Vydac C 18 column (5 μM, 4.6×250 mm), using a gradient of 0.1% TFA in water as solvent A (90-10%) and 0.1% TFA in ACN as solvent B (10-90%), for 40 minutes. As shown in FIG. 1, the crude preparation contains two main peaks (A and B) having retention times of 30.19 and 30.37 minutes, and peak C at 32.92 minutes which derived from the rhenium reagent. Analysis of these peaks by mass spectra indicated (FIG. 2) that the two peaks have the same mass of the expected compound (Re-MAG3-3265) that is 1647 dalton (the expected mass is 1648.52 m/z and the mass of the doubly charged species is 824.65 m/z). The compounds were therefore denoted PTR 3265A (corresponds to peak A in FIG. 1) and PTR 3265B (corresponds to peak B in FIG. 1). Reversed phase HPLC purification of crude yielded 1.49 mg PTR 3265A (first to elute) and 1.21 mg PTR 3265B.

### Example 4

### Binding of Analogs to Somatostatin Receptors.

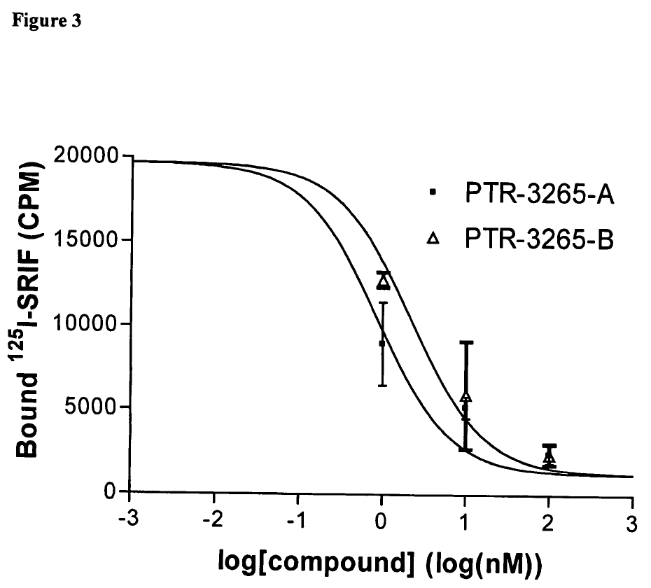

The ability of the SST analogs of the invention to bind to SST receptors in vitro was demonstrated by assaying the ability of such analogs to inhibit binding of a radiolabelled SST analog to SST receptor-containing cell membranes as described above. The receptor membrane preparations used for these tests were from the cloned human receptors selectively and stably expressed in CHO cells and the radiolabelled analog used was (3-[¹²⁵I]tyrosyl¹¹)SRIF-14. Table No. 3 describes the results of the binding assays of PTR 3161, 3265A and 3265B to the human cloned SST-R2 while FIG. 3 describes the competitive binding curves of 3265A and 3265B.

TABLE NO 3

| Peptide | Total Bound (3-[¹²⁵I]tyrosyl11)somatostatin-14 (cpm) | | |
|---|---|---|---|
| conc. (nM) | Run 1 | Run 2 | Run 3 |
| PTR 3261 | | | |
| 10000 | 917 | 1106 | 2281 |
| 1000 | 1941 | 3169 | 2024 |
| 100 | 5398 | 5328 | 5679 |
| 10 | 8335 | 8820 | 8098 |
| PTR 3265A | | | |
| 100 | 2461 | 2222 | 2642 |
| 10 | 5077 | 4978 | 5463 |
| 1 | 9819 | 9257 | 7867 |
| PTR 3265B | | | |
| 100 | 2157 | 2480 | 2628 |
| 10 | 4444 | 6617 | 6738 |
| 1 | 12740 | 12682 | 13026 |

The data presented in the above table show that the peptides of the instant invention have a high affinity of binding for SST receptors. The IC₅₀ values calculated from these experimental results and are:

The IC₅₀ of PTR 3261 is 118.8 nM (95% confidence interval 84.7-166.6 nM). For PTR 3265A the IC₅₀ is 0.8532 nM (95% confidence interval 0.5185-1.404 nM). For PTR 3265B the IC₅₀ is 2.156 nM (95% confidence interval 1.487-3.126 nM).

### Example 5

### Synthesis of PTR 3271A and 3271B (the oxo[^{99m}Tc]technetium-PTR 3261 Isomeric Pair)

To a “cold kit” vial containing PTR-3261 (26.6 μg, 18.4 nmol), sodium glucoheptanoate (2.33 mg, 9.40 μmol), stannous chloride (21.0 μg, 111 nmol) and ethylenediaminetetraacetic and (39.5 μg, 135 nmol) is added saline eluent from a ⁹⁹Mo/^{99m}Tc generator (1 mL, 100 mCi sodium [^{99m}Tc]pertechnetate). The vial is heated at 100° C. for 10 min to afford the reduction of technetium from the +7 oxidation state to the +5 oxidation state, the transient formation of the “weak” technetium-glucoheptanoate complex and the transchelation of technetium to form the “strong” complex, namely the PTR 3271A and 3271 B isomeric mixture.

The radiochemical purity of the mixture is checked via radiometric thin layer chromatography and HPLC and the chemical purity of the mixture is checked via HPLC. The rhenium-containing surrogate isomeric mixture, PTR 3265A and 3265B, serves as the reference standard.

The product mixture (“cold” PTR 3261 with “hot” PTR 3271A and 3271B) can be used directly in in vitro and in vivo studies (i.e., emulating the product that would be used in the nuclear medicine clinic). Alternatively, using semi-preparative HPLC, the “hot” PTR 3271 isomeric mixture can be separated from the “cold” PTR-3261 for such biological studies. Another alternative is HPLC separation of the individual isomers, PTR 3271 A and PTR 3271B for use in biological studies. In each alternative, the radiochemical purity of the mixture is checked via radiometric thin layer chromatography and HPLC and the chemical purity of the mixture is checked via HPLC.

### Example 6

### Localization and In Vivo Imaging of SST-R—Expressing Tumors in Rats.

In vivo imaging of SST receptors expressed by rat tumor cells is performed essentially as described by Bakker et al. (1991, Life Sciences 42:1593-1601). Tumor cells are implanted intramuscularly in a suspension of 0.05 to 0.1 mL/animal, into the right hind thigh of 6 week old rats. The tumors are allowed to grow to approximately 0.5 to 2 g, harvested, and tumor brei is used to implant a second, naive set of Lewis rats. Passaging in this fashion is repeated to generate successive generations of tumor-bearing animals. The tumor-bearing animals used for the in vivo studies are usually from the third to fifth passage and carried 0.2 to 2 g tumors. For studies of the specificity of radiotracer localization in the tumors, selected animals are given an subcutaneous SST-R blocking dose (4 mg/kg) of Octreotide 30 minutes prior to injection of the radiotracer. (This protocol has been shown by Bakker et al. to result in a lowering of ¹¹¹In-DTPA-Octreotide tumor uptake by 40%). Third- to fifth-passage tumor-bearing rats are injected intravenously via the dorsal tail vein with a dose of 0.15-0.20 mCi 99 mTc-labeled compound corresponding to 3 to 8 μg peptide in 0.2 to 0.4 mL. At selected times, the animals are sacrificed by cervical dislocation and harvested tissue samples are weighed and counted along with an aliquot of the injected dose in a gamma well-counter.

### Example 7

### Additional Derivatives of PTR 3261

Six additional compounds which are derivatives of the sequence Phe(C3**)-Cys*-Phe-(D)Trp-Lys-Thr-Cys*-Phe(N3**)-NH2, comprising N₃S or N₂S₂-type chelators, were synthesized with and without metal labeling. The compounds are described in table 4 below.

TABLE 4

| PTR | Sequence |
|---|---|
| 3303 | MA-Dap(MA)-Gly-Phe(C3**)-Cys*-Phe-(D)Trp-Lys-Thr-Cys*-Phe(N3**)-NH₂ |
| 3305 | MA-Dap(Gly)-Gly-Phe(C3**)-Cys*-Phe-(D)Trp-Lys-Thr-Cys*-Phe(N3**)-NH₂ |
| 3307 | Gly-Dap(MA)-Gly-Phe(C3**)-Cys*-Phe-(D)Trp-Lys-Thr-Cys*-Phe(N3**)-NH₂ |
| 3309 | MA-Dap(MA)-GABA-Phe(C3**)-Cys*-Phe-(D)Trp-Lys-Thr-Cys*-Phe(N3**)-NH₂ |
| 3311 | MA-Dap(Gly)-GABA-Phe(C3**)-Cys*-Phe-(D)Trp-Lys-Thr-Cys*-Phe(N3**)-NH₂ |
| 3313 | Gly-Dap(MA)-GABA-Phe(C3**)-Cys*-Phe-(D)Trp-Lys-Thr-Cys*-Phe(N3**)-NH₂ |

Five additional analogs of PTR-3261 comprising DTPA as bi-functional chelator were made. The compounds were tested for inhibition of SRIF binding to the SST-R2 and the results are summarized in the following table 5.

TABLE 5

| | | SRIF Inhibited (%) | |
|---|---|---|---|
| PTR | Sequence | 100 nM | 10 nM |
| 3347 | In-DTPA-Gly-Phe(C3**)-Cys*-Phe- | 51 | 38 |
| | (D)Trp-Lys-Thr-Cys*-Phe(N3**)-NH2 | | |
| 3349 | In-DTPA-b-Ala-Phe(C3**)-Cys*-Phe- | 54 | 20 |
| | (D)Trp-Lys-Thr-Cys*-Phe(N3**)-NH2 | | |
| 3351 | In-DTPA-GABA-Phe(C3**)-Cys*-Phe- | 53 | 5 |
| | (D)Trp-Lys-Thr-Cys*-Phe(N3 **)-NH2 | | |
| 3353 | In-DTPA-5-aminopentanoic acid-Phe(C3**)- | 59 | 14 |
| | Cys*-Phe-(D)Trp-Lys-Thr-Cys*- | | |
| | Phe(N3**)-NH2 | | |
| 3355 | In-DTPA-3-aminomethylbenzoic acid- | 34 | 8 |
| | Phe(C3**)-Cys*-Phe-(D)Trp-Lys-Thr- | | |
| | Cys*-Phe(N3**)-NH2 | | |

### Example 8

### Backbone Cyclized Somatostatin Analogs of PTR 3173

The compound denoted PTR 3173 is a backbone cyclized somatostatin analog selective for SST-R2 and SST-R5. Its synthesis and activity are described in WO 99/65508. The compound has the following structure:

PTR 3173 was chosen as a lead for radiolabeling due to its chemical and pharmacological properties. In addition to subtype 2 selectivity that would allow detection of most neuroendocrine-related tumors, it could detect those derived from the pituitary (expressing hsstr5 predominantly). The core peptide is less lipophilic, much easier to synthesize and the sugar derivative, PTR-3229, retains the potency of the parent, suggesting a good site for conjugation of chelators for radiometals.

Linkers/spacers were used to understand the relationship of the distance of the sometimes bulky, sometimes charged metal core to the phamacophore. Additionally, rigidity/flexibility of the linkers/spacers was studied.

First set of analogs synthesized, purified, characterized and tested for binding to SST-Rs, which found to exhibit high affinity to SST-R2 and SST-R5:

PTR 3249: 1,3-dicarbonyl-Cyclopropane*-Phe-Trp-(D)Trp-Lys-Thr-Phe-GlyN3-NH₂;

PTR 3255: Phthalic acid*-Phe-Trp-(D)Trp-Lys-Thr-Phe-GlyN3-NH₂;

PTR 3257: Maleic acid*-Phe-Trp-(D)Trp-Lys-Thr-Phe-GlyN3-NH₂;

The asterisk denotes that the bridging group is connected between the free functional group of that residue the N^{α}-ω-functionalized derivative of the Gly residue.

Second set of analogs:

PTR 3251: 1,3-dicarbonyl-Cyclopropane*-Tyr-Trp-(D)Trp-Lys-Thr-Phe-GlyN3-NH₂

PTR 3253: Glutanic acid*-Phe-Trp-(D)Trp-Lys-Thr-Phe-GlyN3-NH₂

PTR 3239: Diaminoethane*-CO-Phe-Trp-(D)Trp-Lys-Thr-Phe-GlyC3-NH₂

PTR 3241: Diaminoethane*-CO-Tyr-Trp-(D)Trp-Lys-Thr-Phe-GlyC3-NH₂

PTR 3245: Diaminopropan*-CO-Phe-Trp-(D)Trp-Lys-Thr-Phe-GlyC3-NH₂

PTR 3247: Diaminoethane*-CO-Phe-Trp-(D)Trp-Lys-Thr-Phe-GlyC3-NH₂

PTR 3263: GABA*-Phe-Trp-(D)Trp-(D)Lys-Thr-Phe-GlyC3-NH₂

PTR 3243: Valeric acid*-Phe-(D)Trp-GlyC3-Lys-Thr-Phe-NH₂

The asterisk denotes that the bridging group is connected between the free functional group of that residue the N^{α}-ω-functionalized derivative of the Gly residue.

Indium-DTPA Derivatives of PTR 3173: Six backbone cyclized analogs comprising DTPA as bi-functional chelator, were synthesized and tested for inhibition of SRIF binding to SST-R2. The purified metal-free compounds will be used in ¹¹¹In radiochemistry and in in vitro and in vivo studies. The structures and activity are presented in the following table 6.

ReO—-N₂S₂ and —N₃S Derivatives:

19 compounds which are analogs of PTR 3173 labelled with ReO using N₂S₂ or N₃S chelators, were made and screened. A number of the compounds were found to be potent. MPS-30012-02, 30012-08 and 30012-24 were all re-synthesized naked, purified and radiolabelled with ^{99m}Tc. In vitro binding studies on all three ^{99m}Tc compounds was performed. 30012-05, 30012-08 and 30012-24 were screened for subtype selectivity analysis. The structures and results are given in table 7.

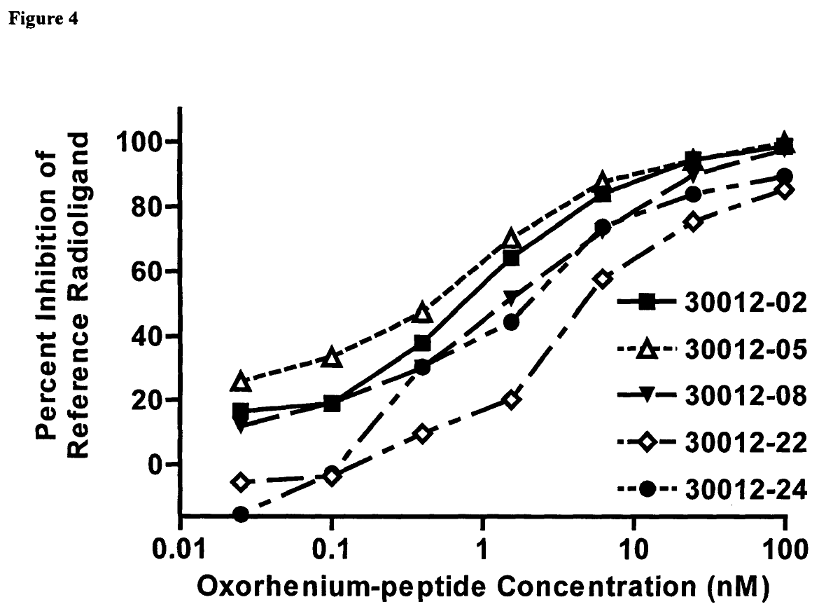

FIG. 4 further demonstrates the affinity of the selected compounds to human SST-R2, measured by inhibition of the reference compound ¹²⁵I-Tyr¹¹-SRIF14.

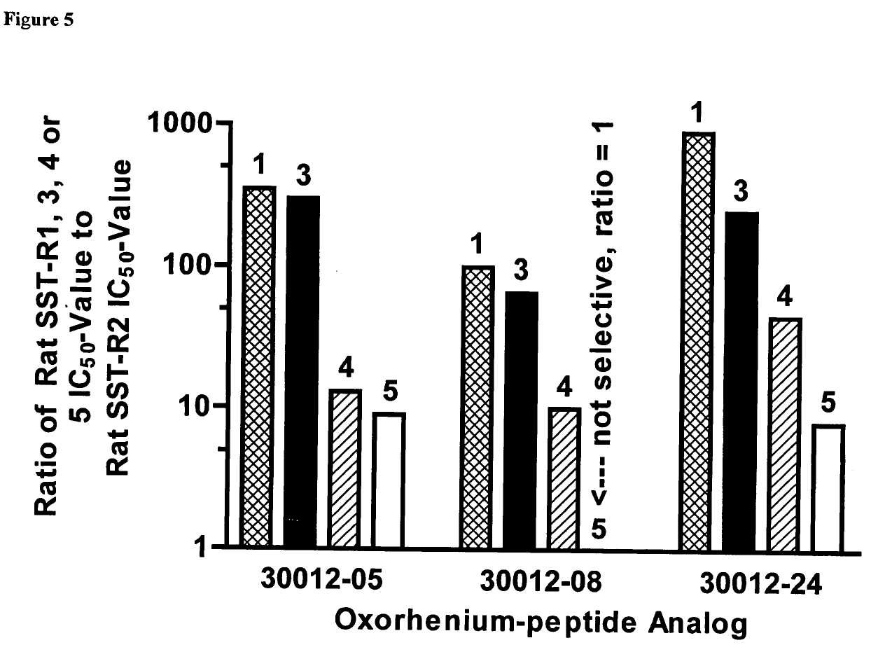

FIG. 5 demonstrates the selectivity of selected compounds to rat SST receptor subtype 2. The ratio value is calculated by dividing the IC₅₀ of the compound in indicated receptor type (1, 3, 4 or 5) by the IC₅₀ of the compound in SST-R2, and thus a larger value indicates a higher selectivity.

* In the below table, 30012-02 denotes also PTR 3395, 30012-05 denotes also ReO-PTR 3397, 30012-08 denotes also PTR 3399, 30012-24 denotes also PTR 3401.

It should be noted that in the three last compounds of table 7 the order of the residues Trp and (D)Trp was reversed relative to the parent molecule PTR-3173 and thus the compounds lost their binding affinity to SST-R2. This indicates the importance of the peptide conformation on the binding activity.

TABLE 6

| | | Quick Screen | | IC₅₀ Screen | | | | | | |
|---|---|---|---|---|---|---|---|---|---|---|
| | | SRIF Inhibited (%) | | SRIF Inhibited (%) | | | | | IC₅₀ | SE log(IC₅₀) |
| Structure | PTR | 100 nM | 10 nM | 100 nM | 10 nM | 1 nM | 0.1 nM | 0.01 nM | (nM) | [log(nM)] |
| In-DTPA-Gly-Dab*-Phe-Trp-(D)Trp-Lys-Thr- | 3319 | 83 | 65 | 86 | 57 | 30 | −0.67 | −8.5 | 7.3 | 0.08 |
| Phe-GlyC3*-NH2 | | | | | | | | | | |
| In-DTPA-Dab*-Phe-Trp-(D)Trp-Lys-Thr-Phe- | 3337 | 87 | 53 | 89 | 69 | 37 | 10 | −8.5 | 4.6 | 0.08 |
| GlyC3*-NH2 | | | | | | | | | | |
| In-DTPA-b-Ala-Dab*-Phe-Trp-(D)Trp-Lys-Thr- | 3339 | 69 | 27 | 78 | 52 | 17 | 3.0 | −11 | 11 | 0.07 |
| Phe-GlyC3*-NH2 | | | | | | | | | | |
| In-DTPA-GABA-Dab*-Phe-Trp-(D)Trp-Lys- | 3341 | 76 | 35 | 76 | 44 | 12 | −4.8 | −9.5 | 16 | 0.09 |
| Thr-Phe-GlyC3*-NH2 | | | | | | | | | | |
| In-DTPA-5-aminopentanoic acid-Dab*-Phe-Trp- | 3343 | 79 | 37 | 76 | 44 | 10 | −3.8 | −16 | −14 | 0.07 |
| (D)Trp-Lys-Thr-Phe-GlyC3*-NH2 | | | | | | | | | | |
| In-DTPA-3-aminomethylbenzoic acid-Dab*-Phe- | 3345 | 80 | 39 | 81 | 55 | 18 | 3.0 | −6.1 | 12 | 0.08 |
| Trp-(D)Trp-Lys-Thr-Phe-GlyC3*-NH2 | | | | | | | | | | |

TABLE 7

| | | hSST- | | | | | |
|---|---|---|---|---|---|---|---|
| | | R2 | Selectivity: IC50, nM, | | | | |
| 30012- | | IC50, | rat SST-R- | | | | |
| no. | Sequence | nM | 1 | 2 | 3 | 4 | 5 |
| 01 | ReO-MA-Dpr(MA)-Dab*-Phe-Trp-(D)Trp-Lys-Thr-Phe-GlyC3*-NH₂ | 12.2 | | | | | |
| 02* | ReO-MA-Dpr(Gly)-Dab*-Phe-Trp-(D)Trp-Lys-Thr-Phe-GlyC3*-NH₂ | 1.14 | | | | | |
| 04 | ReO-MA-Dpr(MA)-Gly-Dab*-Phe-Trp-(D)Trp-Lys-Thr-Phe-GlyC3*-NH₂ | 3.9 | | | | | |
| 05* | ReO-MA-Dpr(Gly)-Gly-Dab*-Phe-Trp-(D)Trp-Lys-Thr-Phe-GlyC3*-NH₂ | 0.97 | 89 | 0.25 | 76 | 3.3 | 2.2 |
| 07 | ReO-MA-Dpr(MA)-βAla-Dab*-Phe-Trp-(D)Trp-Lys-Thr-Phe-GlyC3*-NH₂ | 4 | | | | | |
| 08* | ReO-MA-Dpr(Gly)-βAla-Dab*-Phe-Trp-(D)Trp-Lys-Thr-Phe-GlyC3*-NH₂ | 1.9 | 156 | 1.6 | 103 | 15 | 1.3 |
| 10 | ReO-MA-Dpr(MA)-GABA-Dab*-Phe-Trp-(D)Trp-Lys-Thr-Phe-GlyC3*-NH₂ | 19 | | | | | |
| 11 | ReO-MA-Dpr(Gly)-GABA-Dab*-Phe-Trp-(D)Trp-Lys-Thr-Phe-GlyC3*-NH₂ | >100 | | | | | |
| 13 | ReO-MA-Dpr(MA)-5-aminopentanoic acid-Dab*-Phe-Trp-(D)Trp-Lys-Thr-Phe-GlyC3*-NH₂ | 4.2 | | | | | |
| 16 | ReO-MA-Dpr(MA)-6-aminohexanoic acid-Dab*-Phe-Trp-(D)Trp-Lys-Thr-Phe-GlyC3*-NH₂ | 10 | | | | | |
| 19 | ReO-MA-Gly-Gly-Gly-Dab*-Phe-Trp-(D)Trp-Lys-Thr-Phe-GlyC3*-NH₂ | 7.9 | | | | | |
| 20 | ReO-MA-Gly-Gly-Gly-Gly-Dab*-Phe-Trp-(D)Trp-Lys-Thr-Phe-GlyC3*-NH₂ | 7.8 | | | | | |
| 21 | ReO-MA-Gly-Gly-Gly-βAla-Dab*-Phe-Trp-(D)Trp-Lys-Thr-Phe-GlyC3*-NH₂ | 28.8 | | | | | |
| 22 | ReO-MA-Gly-Gly-Gly-GABA-Dab*-Phe-Trp-(D)Trp-Lys-Thr-Phe-GlyC3*-NH₂ | 2.5 | | | | | |
| 23 | ReO-MA-Gly-Gly-Gly-5-aminopentanoic acid-Dab*-Phe-Trp-(D)Trp-Lys-Thr-Phe-GlyC3*- | 7 | | | | | |
| | NH₂ | | | | | | |
| 24* | ReO-MA-Gly-Gly-Gly-6-aminohexanoic acid-Dab*-Phe-Trp-(D)Trp-Thr-Phe-GlyC3*-NH₂ | 1.45 | 402 | 0.44 | 110 | 20 | 3.7 |

ReO-TY-79-

| | | |
|---|---|---|
| 159-4 | ReO-MA-Dpr(MA)-βAla-Dab*-Phe-(D)Trp-Trp-Lys-Thr-Phe-GlyC3*-NH₂ | ˜100 |
| 161-1 | ReO-Gly-Dpr(MA)-βAla-Dab*-Phe-(D)Trp-Trp-Lys-Thr-Phe-GlyC3*-NH₂ | >100 |
| 161-2 | ReO-MA-Dpr(Gly)-βAla-Dab*-Phe-(D)Trp-Trp-Lys-Thr-Phe-GlyC3*-NH₂ | >100 |

### Example 9

### Reaction of Crude Metal-Free MPS-30012-10 with Rhenium to Yield the Oxorhenium(V) Complex

Crude MPS-30012-10 (6 μmol, based on resin loading) was dissolved in 2 mL DMF and trichlorooxobis(triphenylphosphine)rhenium(V) (6 μmol) was added in 0.9 mL DMF and the mixture was shaken at room temperature for 2 hours. Removal of DMF was achieved by vacuum centrifugation (sample at 40° C.) for about 10 hours and the resulting product was purified by HPLC, yielding the oxorhenium(V) complex of MPS-30012-10.

### Example 10

### Backbone Cyclized Tetra-Peptides

A 96 well plate with tetra-peptides was designed using the pharmacophore based search algorithm (a method described in international application no. WO 00/65467). The peptides were collected from three runs of the algorithm with different pharmacophores. The first pharmacophore is a four point pharmacophore that includes the position of two aromatic rings, a charged amino group and a C alpha atom. Since this is a small pharmacophore a tolerance of 2.5% was used. The second pharmacopore includes the additional C2 atom, and due to the larger size it allows a tolerance of 5%. The third pharmacophore includes the descriptor centers of pharmacophore 1 together with C3 and C4. The tolerance used is 6%. The peptides chosen for synthesis were the ones that after superposing the pharmacophore assignments still presented a good overlap with the reference compound. Also the peptides selected were the basis of small permutations, trying to better target the origin of the activity, e.g. Alanine residues at the C terminus were mutated to glycine. The 96 backbone cyclic tetra-peptide analogs (multiple parallel synthesis plate AL 30008) were tested for binding to SST-Rs. The most active analogs that show over than 50% inhibition, are described in the following table 8:

TABLE NO 8

| Analog No. | R¹ | R² | R³ | R⁴ |
|---|---|---|---|---|
| 17 | Trp | Ala | (D)Trp | LysC4 |
| 20 | (D)Lys | (D)Trp | Phe | (D)PheC4 |
| 22 | (D)Lys | Trp | (D)Phe | (D)AlaC4 |
| 24 | LysC1 | (D)Trp | (D)Pro | (D)TrpN2 |
| 26 | LysC1 | (D)Trp | (D)Ala | PheN3 |
| 48 | LysC2 | Trp | (D)Phe | GlyN2 |
| 56 | (D)LysC2 | (D)Trp | Phe | AlaN2 |
| 57 | (D)LysC2 | (D)Trp | (D)Phe | (D)AlaN2 |
| 58 | (D)LysC2 | (D)Trp | (D)Phe | GlyN2 |
| 59 | (D)LysC2 | (D)Trp | (D)Phe | (D)PheN2 |
| 60 | (D)LysC2 | (D)Trp | (D)Ala | PheN2 |
| 64 | (D)LysC2 | Trp | (D)Phe | AlaN2 |
| 72 | AlaC1 | Lys | (D)Trp | (D)PheN2 |
| 73 | (D)LysN2 | (D)Trp | (D)Phe | (D)AlaC2 |
| 75 | (D)LysN2 | (D)Trp | (D)Phe | (D)PheC2 |
| 80 | (D)LysN2 | Trp | (D)Ala | (D)PheC2 |
| 81 | (D)PheC1 | Ala | (D)Trp | (D)LysN3 |
| 84 | (D)PheC1 | (D)Trp | Lys | (D)AlaN2 |
| 88 | (D)PheC1 | Trp | Pro | LysN3 |
| 89 | (D)LysC3 | (D)Trp | (D)Phe | (D)PheN2 |
| 90 | (D)LysC3 | (D)Trp | (D)Ala | (D)PheN2 |
| 94 | (D)LysC3 | Trp | (D)Phe | (D)PheN2 |
| 96 | (D)LysC3 | Trp | (D)Ala | (D)PheN2 |

All peptide analogs have a C-terminal amide.

Whil the present invention has been described for certain preferred embodiments and examples it will be appreciated by the skilled artisan that many variations and modifications may be performed to optimize the activities of the peptides and analogs of the invention. The examples are to be construed as non-limitative and serve only for illustrative purposes of the principles disclosed according to the present invention, the scope of which is defined by the claims which follow.

What is claimed is:

1. A backbone cyclized analog of somatostatin of three to twenty-four amino acids that incorporates at least one building unit, said building unit containing one nitrogen atom of the peptide backbone connected to a bridging group comprising an amide, thioether, thioester, disulfide, urea, carbamate, or sulfonamide, wherein at least one building unit is connected via the bridging group to form a cyclic structure with a moiety selected from the group consisting of a second building unit, the side chain of an amino acid residue of the sequence or a terminal amino acid residue, further comprising a chelating moiety covalently bound to said backbone cyclized analog.

2. The backbone cyclized analog of claim 1 wherein the chelating moiety is selected from a moiety comprising four donor atoms or eight donor atoms.

3. The somatostatin analog of claim 4 wherein the chelating moiety is selected from 1,4,7,10-tetraazacyclododecane-1,4,7,10-tetraacetic acid (DOTA) and diethylenetriaminepentaacetic acid (DTPA).

4. The somatostatin analog of claim 1 wherein the backbone cyclized somatostatin analog having the general Formula No. 6 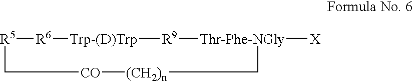
wherein n is 1 to 5;
X designates a terminal carboxy acid, amide or alcohol group;
R⁵ is 1,3-dicarbonyl-cyclopropane, phthalic acid, maleic acid, glutamic acid, valeric acid, diaminoethane-CO, diaminopropane-CO, or GABA;
R⁶ is Phe or Tyr; and
R⁹ is (L)- or (D)-Lys.

5. The backbone cyclized analog of claim 4 selected from the group of:
1,3-dicarbonyl-Cyclopropane*-Tyr-Trp-(D)Trp-Lys-Thr-Phe-GlyN-NH₂;
Glutamic acid*-Phe-Trp-(D)Trp-Lys-Thr-Phe-GlyN3-NH₂;
Diaminoethane*-CO-Phe-Trp-(D)Trp-Lys-Thr-Phe-GlyC3-NH₂;
Diaminoethane*-CO-Tyr-Trp-(D)Trp-Lys-Thr-Phe-GlyC3-NH₂;
Diaminopropan*-CO-Phe-Trp-(D)Trp-Lys-Thr-Phe-GlyC3-NH₂;
Diaminoethane*-CO-Phe-Trp-(D)Trp-Lys-Thr-Phe-GlyC3-NH₂;
GABA*-Phe-Trp-(D)Trp-(D)Lys-Thr-Phe-GlyC3-NH₂;
GABA*-Phe-Trp-(D)Trp-Lys-Thr-Phe-GlyC3-NH₂;
wherein the asterisk denotes that the bridging group is connected between the free functional group of that residue and the N^{α}-ω-functionalized derivative of the Gly residue.

6. The somatostatin analog of claim 1 wherein the backbone cyclized somatostatin analog having the general Formula No. 7 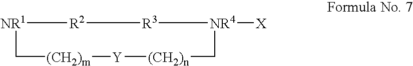
wherein m and n are 1 to 5;
X designates a terminal carboxy acid, amide or alcohol group;
R1 is Trp, (L)- or (D)-Lys, Ala, or Phe;
R2 is Ala, (L)- or (D)-Trp, or Lys
R3 is (D)Trp, (L)- or (D)-Phe, Lys, Pro, or (D)Ala;
R4 is Lys, (D)Phe, (L)- or (D)-Ala, Trp, Gly; and
Y is amide, thioether, thioester, disulfide, urea, carbamate, or sulfonamide.

7. The somatostatin analog of claim 1 wherein the backbone cyclized somatostatin analog having the general Formula No. 8 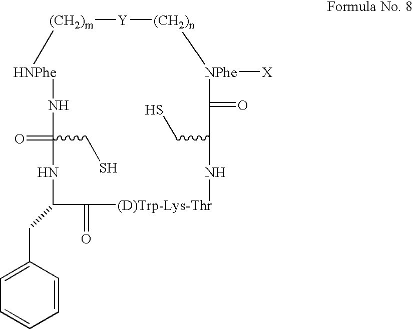
wherein m and n are 1 to 5;
X designates a terminal carboxy acid, amide or alcohol group;
Y is amide, thioether, thioester disulfide, urea, carbamate, or sulfonamide; and
the two cysteine residues are independently L or D.

8. The somatostatin analog of claim 1 having the general Formula No. 5:
Z-Q-PTR Formula No. 5
wherein
Z is a chelating moiety comprising: (i) four donor atoms selected form the group of N₃S and N₂S₂ that through metal complexation form three five- to six-membered rings or (ii) eight donor atoms that, through metal complexation, form stable five- to six-membered rings;
Q is a direct bond or a linker moiety which can be coupled to a free functional group of the peptide; and PTR denotes a backbone cyclized SST analog according to the present invention.

9. The somatostatin analog of claim 8 wherein Q is selected from the group of a direct bond, diaminopropionic acid (Dpr), diaminobutyric acid (Dab), gamma aminobutyric acid (GABA), aminohexanoic acid, polyethylene glycol (PEG), 4-aminobutyric acid, 6-aminocaproic acid, and β-alanine, and Z is selected from the group of mercaptoacetyl-Gly-Gly-Gly (MAG3), 1,4,7,10-tetraazacyclododecane-1,4,7,10-tetraacetic acid (DOTA) and diethylenetriaminepentaacetic acid (DTPA).

10. The somatostatin analog of claim 9 wherein Z is selected from the group of mercaptoacetyl-Gly-Gly-Gly (MAG3), 1,4,7,10-tetraazacyclododecane-1,4,7,10-tetraacetic acid (DOTA) and diethylenetriaminepentaacetic acid (DTPA); and Q is selected from a direct bond and Gly.

11. The somatostatin analog of claim 8 wherein the backbone cyclized somatostatin analog having the general Formula No. 9 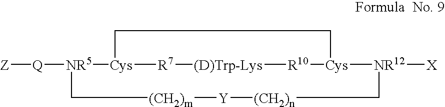
wherein m and n are 1 to 5;
X designates a terminal carboxy acid, amide or alcohol group;
Q is a direct bond or a linker moiety which can be coupled to any free functional group of the peptide;
Z is a chelating moiety comprising: (i) four donor atoms selected form the group of N₃S and N₂S₂ that through metal complexation form 5- to 6-membered rings or (ii) eight donor atoms that, through metal complexation, form stable five- to six-membered rings;
R⁵ is (D)- or (L)-Phe or (D)- or (L)-Ala;
R⁷ is (D)- or (L)-Trp, (D)- or (L)-Phe, (D)- or (L)-1Nal, or (D)- or (L)-2Nal, or (D)- or (L) Tyr;
R¹⁰ is Thr, Gly, Abu, Ser, Cys, Val, (D)- or (L)-Ala, or (D)- or (L)-Phe;
R¹² is (D)- or (L)-Phe or (D)- or (L)-Ala; and
Y is amide, thioether, thioester disulfide, urea, carbamate, or sulfonamide.

12. The somatostatin analog of claim 11 wherein the backbone cyclized somatostatin analog is selected from the group of:
MAG3-PheC3**-Cys*-Phe-(D)Trp-Lys-The-Cys*-PheN3**-NH₂;
MA-Dap(MA)-Gly-Phe(C3**)-Cys*-Phe-(D)Trp-Lys-Thr-Cys*-Phe(N3**)-NH₂;
MA-Dap(Gly)-Gly-Phe(C3**)-Cys*-Phe-(D)Trp-Lys-Thr-Cys*-Phe(N3**)-NH₂;
Gly-Dap(MA)-Gly-Phe(C3**)-Cys*-Phe-(D)Trp-Lys-Thr-Cys*-Phe(N3**)-NH₂;
MA-Dap(MA)-GABA-Phe(C3**)-Cys*-Phe-(D)Trp-Lys-Thr-Cys*-Phe(N3**)-NH₂;
MA-Dap(Gly)-GABA-Phe(C3**)-Cys*-Phe-(D)Trp-Lys-Thr-Cys*-Phe(N3**)-NH₂;
Gly-Dap(MA)-GABA-Phe(C3**)-Cys*-Phe-(D)Tp-Lys-Thr-Cys*-Phe(N3**)-NH₂;
wherein MA denotes mercaptoacetyl, Dpr denotes diaminopropionic acid, Dab denotes diaminobutyric acid, GABA denotes gamma aminobutyric acid, one asterisk denotes that the two cysteines form a disulfide bridge, and two asterisks denotes that a second bridging group is connected between the N^{α}-ω-functionalized derivative of marked residues.

13. The somatostatin analog of claim 8 wherein the backbone cyclized somatostatin analog having the general Formula No. 10 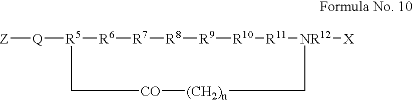
wherein n is 1 to 5;
X designates a terminal carboxy acid, amide or alcohol group;
Z is a chelating moiety comprising: (i) four donor atoms selected form the group of N₃S and N₂S₂ that, through metal complexation, form three 5- to 6-membered rings or (ii) eight donor atoms that, through metal complexation, form stable five- to six-membered rings;
Q is a direct bond or a linker moiety which can be coupled to a free functional group of the peptide;
R⁵ is diaminobutyric acid or diaminopropionic acid;
R⁶ is (D)- or (L)-Phe or Tyr;
R⁷ is (D)- or (L)-Trp, (D)- or (L)-Phe, (D)- or (L)-1Nal or (D)- or (L)-2Nal, or Tyr;
R⁸ is (D)- or (L)-Trp;
R⁹ is (D)- or (L)-Lys;
R10 is Thr, Gly, Abu, Ser, Cys, Val, (D)- or (L)-Ala, or (D)- or (L)-Phe;
R¹¹ is (D)- or (L)-Phe, (D)- or (L)-Ala, Nle, or Cys; and
R¹² is Gly, Val, Leu, (D)- or (L)-Phe or 1Nal or 2Nal.

14. The somatostatin analog of claim 13 wherein:
X is amide;
Q is selected from the group of a direct bond, Gly, P-Ala and GABA;
Z is DOTA or DTPA;
R⁵ is diaminobutyric acid (Dab);
R⁶ is Phe;
R⁷ is Trp;
R⁸ is (D)Trp;
R⁹ is Lys;
R¹⁰ is Thr;
R¹¹ is Phe;
R¹² is Gly; and
n is 3.

15. The somatostatin analog of claim 13 wherein the backbone cyclized somatostatin analog is selected from the group of:
In-DTPA-Gly-Dab*-Phe-Trp-(D)Trp-Lys-Thr-Phe-GlyC3*-NH₂;
In-DTPA-Dab*-Phe-Trp-(D)Trp-Lys-Thr-Phe-GlyC3*-NH₂;
In-DTPA-β-Ala-Dab*-Phe-Trp-(D)Trp-Lys-Thr-Phe-GlyC3*-NH₂;
In-DTPA-GABA-Dab*-Phe-Trp-(D)Trp-Lys-Thr-Phe-GlyC3*-NH₂;
In-DTPA-5-aminopentanoic acid-Dab*-Phe-Trp-(D)Trp-Lys-Thr-Phe-GlyC3*-NH₂;
In-DTPA-3-aminomethylbenzoic acid-Dab*-Phe-Trp-(D)Trp-Lys-Thr-Phe-GlyC3*-NH₂;
ReO-MA-Dpr(MA)-Dab*-Phe-Trp-(D)Trp-Lys-Thr-Phe-GlyC3*-NH₂;
ReO-MA-Dpr(MA)-Gly-Dab*-Phe-Trp-(D)Trp-Lys-Thr-Phe-GlyC3*-NH₂;
ReO-MA-Dpr(MA)-βAla-Dab*-Phe-Trp-(D)Trp-Lys-Thr-Phe-GlyC3*-NH₂;
ReO-MA-Dpr(MA)-GABA-Dab*-Phe-Trp-(D)Trp-Lys-Thr-Phe-GlyC3*-NH₂;
ReO-MA-Dpr(MA)-5-aminopentanoic acid-Dab*-Phe-Trp-(D)Trp-Lys-Thr-Phe-GlyC3*-NH₂;
ReO-MA-Dpr(MA)-6-aminohexanoic acid-Dab*-Phe-Trp-(D)Trp-Lys-Thr-Phe-GlyC3*-NH₂;
ReO-MA-Gly-Gly-Gly-Dab*-Phe-Trp-(D)Trp-Lys-Thr-Phe-GlyC3*-NH2;
ReO-MA-Gly-Gly-Gly-Gly-Dab*-Phe-Trp-(D)Trp-Lys-Thr-Phe-GlyC3*-NH₂;
ReO-MA-Gly-Gly-Gly-GABA-Dab*-Phe-Trp-(D)Trp-Lys-Thr-Phe-GlyC3*-NH₂;
ReO-MA-Gly-Gly-Gly-5-aminopentanoic acid-Dab*-Phe-Trp-(D)Trp-Lys-Thr-Phe-GlyC3*-NH₂;
wherein MA denotes mercaptoacetyl, Dpr denotes diaminopropionic acid, Dab denotes diaminobutyric acid, GABA denotes gamma aminobutyric acid, and the asterisk denotes that the bridging group is connected between the free functional group of that residue and the N^{α}-ω-functionalized derivative of the Gly residue.

16. The backbone cyclized analog of claim 1 wherein the chelating moiety further comprises a complex with a radioisotope.

17. The somatostatin analog according to claim 16 wherein the radioisotope is selected from the group consisting of ^{99m}Tc, ¹⁸⁶Re, ¹⁸⁸Re, indium, yttrium, lutetium, gallium and gadolinium.

18. A method for diagnosing or treating cancer and allograft rejection comprising administration of a backbone cyclized analog of somatostatin of three to twenty-four amino acids that incorporates at least one building unit, said building unit containing one nitrogen atom of the peptide backbone connected to a bridging group comprising an amide, thioether, thioester, disulfide, urea, carbamate, or sulfonamide, wherein at least one building unit is connected via the bridging group to form a cyclic structure with a moiety selected from the group consisting of a second building unit, the side chain of an amino acid residue of the sequence or a terminal amino acid residue; a chelating moiety covalently bound to said backbone cyclized analog; and a pharmaceutically acceptable carrier.

19. The method according to claim 18 wherein the backbone cyclic analog is used for imaging metastases.

20. The method according to claim 18 wherein the backbone cyclic analog is labeled with a detectable tracer.

21. The method according to claim 18 wherein the chelating moiety is selected from 1,4,7,10-tetraazacyclododecane-1,4,7,10-tetraacetic acid (DOTA) and diethylenetriaminepentaacetic acid (DTPA).

22. The method according to claim 18 wherein the backbone cyclized somatostatin analog having the general Formula No. 6 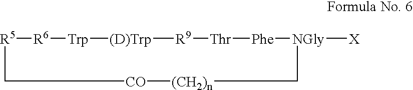
wherein n is 1 to 5;
X designates a terminal carboxy acid, amide or alcohol group;
R⁵ is 1,3-dicarbonyl-cyclopropane, phthalic acid, maleic acid, glutamic acid, valeric acid, diaminoethane-CO, diaminopropane-CO, or GABA;
R⁶ is Phe or Tyr; and
R⁹ is (L)- or (D)-Lys.

23. The method according to claim 22 selected from the group of:
1,3-dicarbonyl-Cyclopropane*-Tyr-Trp-(D)Trp-Lys-Thr-Phe-GlyN3-NH₂;
Glutamic acid*-Phe-Trp-(D)Trp-Lys-Thr-Phe-GlyN3-NH₂;
Diaminoethane*-CO-Phe-Trp-(D)Tp-Lys-Thr-Phe-GlyC3-NH₂;
Diaminoethane*-CO-Tyr-Trp-(D)Trp-Lys-Thr-Phe-GlyC3-NH₂;
Diaminopropan*-CO-Phe-Trp-(D)Trp-Lys-Thr-Phe-GlyC3-NH₂;
Diaminoethane*-CO-Phe-Trp-(D)Trp-Lys-Thr-Phe-GlyC3-NH₂;
GABA*-Phe-Trp-(D)Trp-(D)Lys-Thr-Phe-GlyC3-NH₂;
GABA*-Phe-Trp-(D)Trp-Lys-Thr-Phe-GlyC3-NH₂;
wherein GABA denotes gamma aminobutyric acid, and the asterisk denotes that the bridging group is connected between the free functional group of that residue and the N^{α}-ω-functionalized derivative of the Gly residue.

24. The method according to claim 18 wherein the backbone cyclized somatostatin analog having the general Formula No. 7 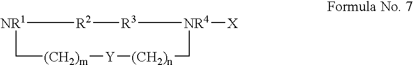
wherein m and n are 1 to 5;
X designates a terminal carboxy acid, amide or alcohol group;
R1 is Trp, (L)- or (D)-Lys, Ala, or Phe;
R2 is Ala, (L)- or (D)-Trp, or Lys;
R3 is (D)Trp, (L)- or (D)-Phe, Lys, Pro, or (D)Ala;
R4 is Lys, (D)Phe, (L)- or (D)-Ala, Tip, Gly; and
Y is amide, thioether, thioester, disulfide, urea, carbamate, or sulfonamide.

25. The method according to claim 18 wherein the backbone cyclized somatostatin analog having the general Formula No. 8 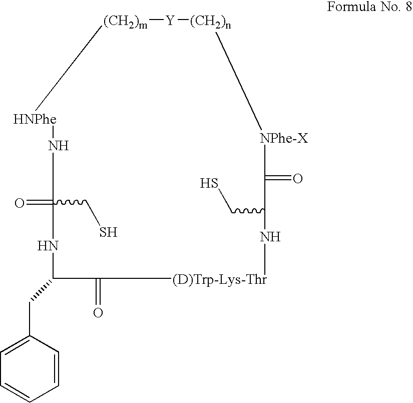
wherein m and n are 1 to 5;
X designates a terminal carboxy acid, amide or alcohol group;
Y is amide, thioether, thioester disulfide, urea, carbamate, or sulfonamide; and
the two cysteine residues are independently L or D.

26. The method according to claim 18 having the general Formula No. 5:
Z-Q-PTR Formula No. 5
wherein
Z is a chelating moiety comprising: (i) four donor atoms selected form the group of N₃S and N₂S₂ that through metal complexation form three five- to six-membered rings or (ii) eight donor atoms that, through metal complexation, form stable five- to six-membered rings;
Q is a direct bond or a linker moiety which can be coupled to a free functional group of the peptide; and PTR denotes a backbone cyclized SST analog according to the present invention.

27. The method according to claim 26 wherein Q is selected from the group of a direct bond, diaminopropionic acid (Dpr), diaminobutyric acid (Dab), gamma aminobutyric acid (GABA), aminohexanoic acid, polyethylene glycol (PEG), 4-aminobutyric acid, 6-aminocaproic acid, and p-alanine, and Z is selected from the group of mercaptoacetyl-Gly-Gly-Gly (MAG3), 1,4,7,10-tetraazacyclododecane-1,4,7,10-tetraacetic acid (DOTA) and diethylenetriaminepentaacetic acid (DTPA).

28. The method according to claim 27 wherein Z is selected from the group of mercaptoacetyl-Gly-Gly-Gly (MAG3), 1,4,7,10-tetraazacyclododecane-1,4,7,10-tetraacetic acid (DOTA) and diethylenetriaminepentaacetic acid (DTPA); and Q is selected from a direct bond or Gly.

29. The method according to claim 26 wherein the backbone cyclized somatostatin analog having the general Formula No. 9 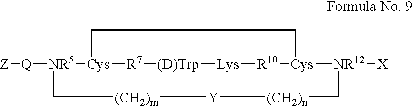
wherein m and n are 1 to 5;
X designates a terminal carboxy acid, amide or alcohol group;
Q is a direct bond or a linker moiety which can be coupled to any free functional group of the peptide;
Z is a chelating moiety comprising: (i) four donor atoms selected form the group of N₃S and N₂S₂ that through metal complexation form 5- to 6-membered rings or (ii) eight donor atoms that, through metal complexation, form stable five- to six-membered rings;
R⁵ is (D)- or (L)-Phe or (D)- or (L)-Ala;
R⁷ is (D)- or (L)-Trp, (D)- or (L)-Phe, (D)- or (L)-1Nal, or (D)- or (L)-2Nal, or (D)- or (L) Tyr;
R10 is Thr, Gly, Abu, Ser, Cys, Val, (D)- or (L)-Ala, or (D)- or (L)-Phe;
R¹² is (D)- or (L)-Phe or (D)- or (L)-Ala; and
Y is amide, thioether, thioester disulfide, urea, carbamate, or sulfonamide.

30. The method according to claim 29 wherein the backbone cyclized somatostatin analog is selected from the group of:
MAG3-PheC3**-Cys*-Phe-(D)Trp-Lys-The-Cys*-PheN3**-NH₂;
MA-Dap(MA)-Gly-Phe(C3**)-Cys*-Phe-(D)Trp-Lys-Thr-Cys*-Phe(N3**)-NH₂;
MA-Dap(Gly)-Gly-Phe(C3**)-Cys*-Phe-(D)Trp-Lys-Thr-Cys*-Phe(N3**)-NH₂;
Gly-Dap(MA)-Gly-Phe(C3**)-Cys*-Phe-(D)Trp-Lys-Thr-Cys*-Phe(N3**)-NH₂;
MA-Dap(MA)-GABA-Phe(C3**)-Cys*-Phe-(D)Trp-Lys-Thr-Cys*-Phe(N3**)-NH₂;
MA-Dap(Gly)-GABA-Phe(C3**)-Cys*-Phe-(D)Trp-Lys-Thr-Cys*-Phe(N3**)-NH₂;
Gly-Dap(MA)-GABA-Phe(C3**)-Cys*-Phe-(D)Trp-Lys-Thr-Cys*-Phe(N3**)-NH₂;
wherein MA denotes mercaptoacetyl, Dab denotes diaminobutyric acid, GABA denotes gamma aminobutyric acid, and one asterisk denotes that the two form a disulfide bridge, and two asterisks denotes that a second bridging group is connected between the N^{α}-ω-functionalized derivative of marked residues.

31. The method according to claim 26 wherein the backbone cyclized somatostatin analog having the general Formula No. 10 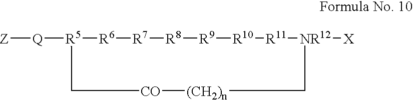
wherein n is 1 to 5;
X designates a terminal carboxy acid, amide or alcohol group;
Z is a chelating moiety comprising: (i) four donor atoms selected form the group of N₃S and N₂S₂ that, through metal complexation, form three 5- to 6-membered rings or (ii) eight donor atoms that, through metal complexation, form stable five- to six-membered rings;
Q is a direct bond or a linker moiety which can be coupled to a free functional group of the peptide;
R⁵ is diaminobutyric acid or diaminopropionic acid.
R⁶ is (D)- or (L)-Phe or Tyr;
R⁷ is (D)- or (L)-Trp, (D)- or (L)-Phe, (D)- or (L)-1Nal or (D)- or (L)-2Nal, or Tyr;
R⁸ is (D)- or (L)-Trp;
R⁹ is (D)- or (L)-Lys;
R10 is Thr, Gly, Abu, Ser, Cys, Val, (D)- or (L)-Ala, or (D)- or (L)-Phe;
R¹¹ is (D)- or (L)-Phe, (D)- or (L)-Ala, Nle, or Cys; and
R¹² is Gly, Val, Leu, (D)- or (L)-Phe or 1Nal or 2Nal.

32. The method according to claim 31 wherein:
X is amide;
Q is selected from the group of a direct bond, Gly, P-Ala and GABA;
Z is DOTA or DTPA;
R⁵ is diaminobutyric acid (Dab);
R⁶ is Phe;
R⁷ is Trp;
R⁸ is (D)Trp;
R⁹ is Lys;
R10 is Thr;
R¹¹ is Phe;
R¹² is Gly; and
n is 3.

33. The method according to claim 31 wherein the backbone cyclized somatostatin analog is selected from the group of:
In-DTPA-Gly-Dab*-Phe-Trp-(D)Trp-Lys-Thr-Phe-GlyC3*-NH₂;
In-DTPA-Dab*-Phe-Trp-(D)Trp-Lys-Thr-Phe-GlyC3*-NH₂;
In-DTPA-P-Ala-Dab*-Phe-Trp-(D)Trp-Lys-Thr-Phe-GlyC3*-NH₂;
In-DTPA-GABA-Dab*-Phe-Trp-(D)Trp-Lys-Thr-Phe-GlyC3*-NH₂;
In-DTPA-5-aminopentanoic acid-Dab*-Phe-Trp-(D)Trp-Lys-Thr-Phe-GlyC3*-NH₂;
In-DTPA-3-aminomethylbenzoic acid-Dab*-Phe-Trp-(D)Trp-Lys-Thr-Phe-GlyC3*-NH₂;
ReO-MA-Dpr(MA)-Dab*-Phe-Trp-(D)Trp-Lys-Thr-Phe-GlyC3*-NH₂;
ReO-MA-Dpr(MA)-Gly-Dab*-Phe-Trp-(D)Trp-Lys-Thr-Phe-GlyC3*-NH₂;
ReO-MA-Dpr(MA)-Ala-Dab*-Phe-Trp-(D)Trp-Lys-Thr-Phe-GlyC3*-NH₂;
ReO-MA-Dpr(MA)-GABA-Dab*-Phe-Trp-(D)Trp-Lys-Thr-Phe-GlyC3*-NH₂;
ReO-MA-Dpr(MA)-5-aminopentanoic acid-Dab*-Phe-Trp-(D)Trp-Lys-Thr-Phe-GlyC3*-NH₂;
ReO-MA-Dpr(MA)-6-aminohexanoic acid-Dab*-Phe-Trp-(D)Trp-Lys-Thr-Phe-GlyC3*-NH₂;
ReO-MA-Gly-Gly-Gly-Dab*-Phe-Trp-(D)Trp-Lys-Thr-Phe-GlyC3*-NH₂;
ReO-MA-Gly-Gly-Gly-Gly-Dab*-Phe-Trp-(D)Trp-Lys-Thr-Phe-GyC3*-NH₂;
ReO-MA-Gly-Gly-Gly-GABA-Dab*-Phe-Trp-(D)Trp-Lys-Thr-Phe-GlyC3*-NH₂;
ReO-MA-Gly-Gly-Gly-5-aminopentanoic acid-Dab*-Phe-Trp-(D)Trp-Lys-Thr-Phe-GlyC3*-NH₂;
wherein MA denotes mercaptoacetyl, Dpr denotes diaminopropionic acid, Dab denotes diaminobutyric acid, GABA denotes gamma aminobutyric acid, DTPA denotes diethylenetriaminepentaacetic acid, and the asterisk denotes that the bridging group is connected between the free functional group of that residue and the N-(o-functionalized derivative of the Gly residue.

34. The method according to claim 18 wherein the chelating moiety further comprises a complex with a radioisotope.

35. The method according to claim 34 wherein the radioisotope is selected from the group consisting of ^{99m}Tc, ¹⁸⁶Re, ⁸⁸Re, indium, yttrium, lutetium, gallium and gadolinium.

36. The method according to claim 18 wherein the backbone cyclic analog is selective for at least one somatostatin receptor subtype.

37. A method for diagnosing, treating or preventing of disorders selected from the group consisting of cancers, autoimmune diseases, endocrine disorders, diabetes-associated complications, gastrointestinal disorders, inflammatory diseases, pancreatitis, atherosclerosis, restenosis, allograft rejection, and post-surgical pain, comprising administering to a mammal in need thereof a pharmaceutical composition comprising a therapeutically effective amount of a backbone cyclized somatostatin analog according to claim 1.

38. The method according to claim 37 wherein the backbone cyclic analog is selective for at least one somatostatin receptor subtype.

39. A kit for preparing a scintigraphic imaging agent for imaging sites within a mammalian body, said kit comprising a backbone cyclized analog of somatostatin and a chelating moiety covalently bound to said backbone cyclized analog.
